(19) United States
(12) Patent Application Publication
(10) Pub. No.: US 2011/0301079 A1
(43) Pub. Date: Dec. 8, 2011

(54) NEUROMEDIN U RECEPTOR AGONISTS AND USES THEREOF

(75) Inventors: Donald J. Marsh, Hillsborough, NJ (US); Antonello Pessi, Rome (IT); Elisabetta Bianchi, Rome (IT); Paolo Ingallinella, Pomezia (Rome) (IT); Andrea Peier, Berkeley Heights, NJ (US); Alessandro Pocai, Westfield, NJ (US)

(73) Assignees: Instituto di Ricerche di Biologia Molecolare P. Angeletti S.P.A.; Merck Sharp & Dohme Corp.

(21) Appl. No.: 12/674,620

(22) PCT Filed: Sep. 17, 2008
(86) PCT No.: PCT/US08/10819

§ 371 Date: Feb. 22, 2010

Related U.S. Application Data
(60) Provisional application No. 60/994,714, filed on Sep. 21, 2007.

(51) Int. Cl. A61K 38/17; C07K 17/02; A61P 3/04; A61P 3/10; A61P 1/16; A61P 9/00; A61P 19/02; A61P 35/00; A61P 3/00; A61P 3/06; C07K 14/47; A61P 9/12

(52) U.S. Cl. 514/5.2; 530/324; 530/391.7; 530/362; 530/380; 514/21.3; 514/6.9; 514/15.7; 514/16.4; 514/16.8; 514/19.3

(57) ABSTRACT

Neuromedin U receptor agonists for use in the treatment of metabolic disorders such as obesity and diabetes are disclosed. In particular, disclosed are neuromedin U receptor agonists that comprise neuromedin S (NMS).

### CROSS-RELATED APPLICATIONS

### Background of the Invention

(1) Field of the Invention

The present invention relates to neuromedin U receptor agonists for use in the treatment of metabolic disorders such as obesity. In particular, the present invention relates to neuromedin U receptor agonists that comprise neuromedin S (NMS).

(2) Description of Related Art

Neuromedin U (NMU) was originally isolated from porcine spinal cord based upon its ability to contract rat uterine smooth muscle and has since been implicated in a variety of other physiological processes, including stress, nociception, inflammation, cardiovascular function and energy homeostasis. Characterization of NMU has identified three peptides with similar bioactivity, full length NMU, (a 25-mer (NMU-25)) in humans, pigs, and dogs, a 23-mer (NMU-23) in rats and mice, and an 8-mer (NMU-8). NMU-8 is derived from cleavage of full-length NMU and shares an identical C-terminus with the full-length precursor. NMU-8 is highly conserved among vertebrates, containing seven C-terminal residues that are identical across all species that have been examined; these residues are critical for bioactivity (Brighton et al., Pharmacol. Rev. 56: 231-248 (2004)).

NMU's role in the regulation of energy homeostasis is supported by both pharmacologic and genetic data. Properties of NMU include inhibition of food intake and increase in energy expenditure seen when the substance is administered centrally (Howard et al., Nature 406: 70-74 (2000); Nakazato et al., Biochem. Biophys. Res. Comm. 277: 191-194 (2000); Ivanov et al., Endocrinol. 143: 3813-3821 (2002); and Wren et al., Endocrinol., 143: 4227-4234 (2002)). NMU-deficient mice develop obesity characterized by hyperphagia and reduced energy expenditure (Hanada et al., Nat. Med., 10: 1067-1073 (2004)), and transgenic mice overexpressing NMU are lean and hypophagic (Kowalski et al., J. Endocrinol. 185: 151-164 (2005)). The internal energy status of an animal affects expression and release of NMU as well (Wren et al., ibid.).

Mori et al. (EMBO J. 24: 325-335 (2005)) recently identified a 36-residue neuropeptide neuromedin S (NMS), which was purified from rat brain extracts. The restricted expression of NMS in the SCN core and the ability of NMS to shift the phase of the circadian rhythm demonstrated that NMS is important for the regulation of circadian rhythm. NMS is a candidate for a nonphotic entrainment factor of circadian rhythm. Ida et al. (Endocrinol. 146: 4217-4223 (2005)) provided evidence suggesting NMS is an anorexigenic hormone. Intracerebroventricular (icy) injection of NMS decreased 12 hour food intake during the dark period in rats. This anorexigenic effect was more potent and persistent than that observed with the same dose of NMU. NMS has also been disclosed in International applications WO2006/086769, WO2007/0221123, and WO2007/075439.

Two high affinity NMU and NMS receptors, NMUR1 (Intl. Patent Appl. No. PCT/US99/15941) and NMUR2 (U.S. Pat. No. 7,163,799), have been identified. NMUR1 is predominantly expressed in the periphery, whereas NMUR2 is primarily expressed in the brain. Pharmacologic experiments have served to better define NMU's short- and long-term effects on energy homeostasis and to identify which NMU receptor(s) are involved in mediating these actions. It has been shown that acute administrations of NMU either centrally or peripherally reduce food intake in mice in a dose-dependent fashion. The anorectic actions of centrally administered NMU are absent in NMUR2-deficient (Nmur2^{-/-}) mice but are present in NMUR1-deficient (Nmur1^{-/-}) mice. In contrast, the anorectic actions of peripherally administered NMU are absent in Nmur1^{-/-} mice and present in Nmur2^{-/-} mice. Additionally, acute peripheral administration of NMU dose-dependently increases core body temperature in mice, suggesting that NMUR1^{-/-} may also modulate energy expenditure. Chronic administration of NMU either centrally or peripherally reduces food intake, body weight and adiposity in mice, again in a dose-dependent fashion. In Nmur2^{-/-} transgenic mice, body weight, body composition, body temperature and food intake are largely unaffected by chronic central administration of rat NMU-23. In Nmur1^{-/-} transgenic mice, body weight, body composition and food intake are largely unaffected by chronic peripheral administration of rat NMU-23.

In light of the above, neuromedin U receptor agonists comprising analogs or derivatives of NMS might be useful in the treatment of metabolic disorders.

### BRIEF SUMMARY OF THE INVENTION

The present invention provides neuromedin U receptor agonists, which comprise derivatives and analogs of neuromedin S (NMS). The neuromedin U receptor agonists can be used therapeutically and as research tools.

Therapeutic applications of the neuromedin U receptor agonists include administering the neuromedin U receptor agonists to an individual to treat a metabolic disorder afflicting the individual. Such disorders include, but are not limited to, obesity, metabolic syndrome or syndrome X, and type II diabetes. Complications of diabetes such as retinopathy may be positively affected thereby as well. Obesity is a comorbidity of and may well contribute to such disease states as diabetes, hypertension, dyslipidemias, cardiovascular disease, gallstones, osteoarthritis and certain forms of cancers. Administration of one or more of the neuromedin U receptor agonists disclosed herein to effect weight loss in an individual may also be useful in preventing such diseases and as part of therapy for any one of the above-recited conditions, as well as others. In other embodiments, there is provided a method for treating a metabolic disease in an individual comprising administering to the individual one or more of the neuromedin U receptor agonists described above. The metabolic disease may be selected from the group consisting of diabetes, metabolic syndrome, hyperglycemia, and obesity and may be administered via a route peripheral to the brain, such as an oral, mucosal, buccal, sublingual, nasal, rectal, subcutaneous, transdermal, intravenous, intramuscular, or intraperitoneal route. Finally, the neuromedin U receptor agonists can be administered to an individual to effect a reduction in food intake by the individual, to effect an increase in energy expenditure by the individual, to effect a reduction in weight gain in the individual, to prevent weight gain in the individual, to effect weight loss in the individual, and/or to prevent weight regain in the individual.

Accordingly, the present invention provides an isolated neuromedin U receptor agonist, which has the formula (I)

Z¹-peptide-Z²
wherein the peptide has the amino acid sequence ILQRG SGTAA VDFTK KDHTA TWGRP FFLFR PRN (SEQ ID NO:1), wherein the peptide can have one or more insertions or substitutions of the amino acid sequence with an alternative amino acid and wherein the peptide can have one or more deletions of the amino acid sequence; Z¹ is an optionally present protecting group that, if present, is joined to the N-terminal amino group; and Z² is NH₂ or an optionally present protecting group that, if present, is joined to the C-terminal carboxy group; and pharmaceutically acceptable salts thereof. In particular aspects, one or more of amino acids 1 to 33 of SEQ ID NO:1 can be a D- or L-amino acid or an alternative amino acid.

In further aspects of the above neuromedin U receptor agonists, the N-terminal amino acid is covalently joined to one or more molecules selected from the group consisting of PEG, cholesterol, N-ethylmaleimidyl, and palmitoyl. In further still aspects of the neuromedin U receptor agonist, the peptide further includes a cysteine residue at the N-terminus of the peptide to which is optionally present a protecting group that, if present, is joined to the N-terminal amino group of the cysteine residue. In particular aspects of the neuromedin U receptor agonist, the thiol group of the cysteine residue at the N-terminus is covalently joined to one or more molecules selected from the group consisting of PEG, cholesterol, N-ethylmaleimidyl, and palmitoyl. In a further embodiment, the neuromedin U receptor agonists has the amino acid of SEQ ID NO:2, which further includes a cysteine residue at the N-terminus of the peptide to which is present a protecting group joined to the N-terminal amino group of the cysteine residue and a PEG molecule joined to the thiol group.

The neuromedin U receptor agonist can further include a linker group having a distal end and a proximal end. The linker group is covalently joined at its distal end to the N-terminus of the peptide, and is covalently linked at the proximal end to the carboxyl terminus of a cysteine residue, onto which is optionally present a protecting group that, if present, is joined to the N-terminal amino group of the cysteine residue. In particular aspects, wherein the thiol group of the cysteine residue is covalently joined to one or more molecules selected from the group consisting of PEG, cholesterol, N-ethylmaleimidyl, and palmitoyl.

The present invention further provides for the use of any one or more of the embodiments and aspects of the neuromedin U receptor agonist in the manufacture of a medicament for treatment of a metabolic disorder. Disorders include, but are not limited to, obesity, metabolic syndrome or syndrome X, and type II diabetes. Complications of diabetes such as retinopathy may be positively affected thereby as well. Obesity is a comorbidity of and may well contribute to such disease states as diabetes, hypertension, dyslipidemias, cardiovascular disease, gallstones, osteoarthritis and certain forms of cancers. Thus, the present invention provides a pharmaceutical composition comprising one or more of any of the above neuromedin U receptor agonists and a pharmaceutically acceptable carrier.

The present invention further provides a method for producing a neuromedin U receptor agonist

In further aspects of the above method, the N-terminal amino acid is covalently joined to one or more molecules selected from the group consisting of PEG, cholesterol, N-ethylmaleimidyl, and palmitoyl. In further still aspects of the neuromedin U receptor agonist, the peptide further includes a cysteine residue at the N-terminus of the peptide to which is optionally present a protecting group that, if present, is joined to the N-terminal amino group of the cysteine residue. In particular aspects of the method, the thiol group of the cysteine residue at the N-terminus is covalently joined to one or more molecules selected from the group consisting of PEG, cholesterol, N-ethylmaleimidyl, and palmitoyl. In a further embodiment, the neuromedin U receptor agonists has the amino acid of SEQ ID NO:1, which further includes a cysteine residue at the N-terminus of the peptide to which is present a protecting group joined to the N-terminal amino group of the cysteine residue and a PEG molecule joined to the thiol group.

The neuromedin U receptor agonist can further include a linker group having a distal end and a proximal end is covalently joined at its distal end to the N-terminus of the peptide and the proximal end of the linker group is covalently linked to the carboxyl terminus of a cysteine residue to which is optionally present a protecting group that, if present, is joined to the N-terminal amino group of the cysteine residue. In particular aspects, wherein the thiol group of the cysteine residue is covalently joined to one or more molecules selected from the group consisting of PEG, cholesterol, N-ethylmaleimidyl, and palmitoyl. In a further embodiment of the method, neuromedin U receptor agonist has the formula Ac—C₂-peptide-CONH₂ wherein Ac is an acetyl group, C2 is Cys(PEG)₂40kDa and the peptide has the amino acid sequence shown in SEQ ID NO:1.

### BRIEF DESCRIPTION OF THE DRAWINGS

FIGS. 1A and 1B show that peripheral administration of NMU and NMS dose-dependently reduced food intake. As shown in FIGS. 1A and 1B, acute peripheral administration of NMS significantly reduced food intake (FIG. 1A) and body weight (FIG. 1B). Also shown are that the anorectic effects of NMS appeared to be greater than the effects observed with NMU.

FIGS. 2A and 2B show that chronic administration of NMS significantly reduced food intake and body weight. The effect was greater than that observed for NMU. Cumulative body weight change (FIG. 2A) and cumulative food intake (FIG. 2B) with two-week treatment of NMU and NMS are shown.

FIGS. 3A, 3B, 3C, and 3D illustrate the anorectic effects of peripherally administered NMS are predominantly mediated by the NMUR1 receptor. Acute peripheral administration of NMS significantly reduced food intake in wild-type mice and Nmur2 knockout mice but not in Nmur1 knockout mice. A modest but significant effect on food intake was observed at the highest dose tested in Nmur1 knockout mice.

FIGS. 4A, 4B, and 4C show that NMS enhances glucose metabolism independently (FIG. 4A, 4B) on its effect on body weight (FIG. 4C).

FIG. 5 shows the pharmacokinetic properties of NMU and NMS in mice: NMS displays greater metabolic stability in vivo. Animals were dosed subcutaneously with 10 mg/kg of hNMU-25 or hNMS. Plasma was collected at various time points post dose and measured in the Bioassay. The dashed line indicates the limits of detection for the assay (LOQ). Consistent with the in vivo data, hNMS exhibits greater metabolic stability compared to NMU.

FIGS. 6A and 6B show that acute subcutaneous administration of PEGylated NMS significantly reduces food intake for at least 3 days post dose. NMS1 and NMS4 can significantly reduce overnight food intake similar to non-PEGylated NMS3. Additionally, PEGylated NMS1 can significantly reduce food intake for 3 days post dose whereas NMS4 only elicited significant reductions in food intake for 2 days post dose FIG. 7A.

FIGS. 7A and 7B show the anorectic effects of PEGylated NMS are comparable in potency to PEGylated NMU. PEGylated NMS and PEGylated NMU (referred to as NMU9) can dose-dependently reduce food intake for at least three days post dose.

FIGS. 8A and 8B show the anorectic effects of NMS1 are mediated by the NMUR1 and NMUR2 receptors. Acute administration of NMS1 was highly efficacious in wild-type animals but no effect on food intake (FIG. 9A) or body weight (FIG. 9B) was observed in the Nmur1/Nmur2 double knockout animals.

FIGS. 9A and 9B show the anorectic effects of PEGylated NMS1 are mediated by both the NMUR1 and NMUR2 receptors. NMS1 effects on food intake are shown in FIG. 9A and NMS1 effects on body weight are shown in FIG. 9B.

FIGS. 10A and 10B show that lipidated analogs of NMS (NMS2 and NMS5) can also reduce food intake and body weight.

### DETAILED DESCRIPTION OF THE INVENTION

The present invention provides neuromedin U receptor agonists, which comprise derivatives and analogs of neuromedin S (NMS). The neuromedin U receptor agonist described herein act at NMU receptors, bind the NMU receptors, and stimulate NMU receptor activity.

One or more of the neuromedin U receptor agonists can be administered to an individual to treat a metabolic disorder afflicting the individual. Such disorders include, but are not limited to, obesity, metabolic syndrome or syndrome X, and type II diabetes. Complications of diabetes such as retinopathy may be positively affected thereby as well. Obesity is a comorbidity of and may well contribute to such disease states as diabetes, hypertension, dyslipidemias, cardiovascular disease, gallstones, osteoarthritis and certain forms of cancers. Administration of one or more of the neuromedin U receptor agonists disclosed herein to effect weight loss in an individual may also be useful in preventing such diseases and as part of therapy for any one of the above-recited conditions, as well as others. In other embodiments, there is provided a method for treating a metabolic disease in an individual comprising administering to the individual a one or more of the neuromedin U receptor agonists described above. The metabolic disease may be selected from the group consisting of diabetes, metabolic syndrome, hyperglycemia, and obesity and may be administered via a route peripheral to the brain, such as an oral, mucosal, buccal, sublingual, nasal, rectal, subcutaneous, transdermal, intravenous, intramuscular, or intraperitoneal route. In particular embodiments, the neuromedin U receptor agonists can be used to treat multiple disorders in an individual. As will be apparent to one of ordinary skill in the art in view of the disclosure herein, the neuromedin U receptor agonists can be administered to an individual to effect a reduction in food intake by the individual, to effect and increase in energy expenditure by the individual, to effect a reduction in weight gain in the individual, to prevent weight gain in the individual, to effect weight loss in the individual, and/or to prevent weight regain in the individual.

Research tool uses may involve the use of a neuromedin U receptor agonist and the presence of an NMU receptor or fragment thereof. Examples of research tool uses include screening for compounds active at NMU receptors, determining the presence of NMU receptors in a sample or preparation, and examining the role or effect of NMU. Additionally, the neuromedin U receptor agonists can be used to screen for NMU binding compounds (agonists or antagonists) by using a neuromedin U receptor agonist in a competition experiment with test compounds.

The neuromedin U receptor agonists of the present invention comprise the general formula (I)

Z¹-peptide-Z²

wherein the peptide has the NMS amino acid sequence ILQRG SGTAA VDFTK KDHTA TWGRP FFLFR PRN (SEQ ID NO:1), wherein the peptide can have one or more insertions or substitutions of the amino acid sequence with an alternative amino acid and wherein the peptide can have one or more deletions of the amino acid sequence; Z¹ is an optionally present protecting group that, if present, is joined to the N-terminal amino group; and Z² is NH₂ or an optionally present protecting group that, if present, is joined to the C-terminal carboxy group; and pharmaceutically acceptable salts thereof.

The phrase “alternative amino acid” as used herein and in the claims encompasses alternative amino acids that are the result of both conservative and non-conservative substitutions. Conservative substitutions are the replacement of an amino acid residue by another similar residue in a polypeptide. Typical but not limiting conservative substitutions are the replacements, for one another, among the aliphatic amino acids Ala, Val, Leu and Ile; interchange of Ser and Thr containing hydroxy residues, interchange of the acidic residues Asp and GIu, interchange between the amide-containing residues Asn and Gln, interchange of the basic residues Lys and Arg, interchange of the aromatic residues Phe and Tyr, and interchange of the small-sized amino acids Ala, Ser, Thr, Met, and Gly. Non-conservative substitutions are the replacement, in a polypeptide, of an amino acid residue by another residue which is not biologically similar. For example, the replacement of an amino acid residue with another residue that has a substantially different charge, a substantially different hydrophobicity, or a substantially different spatial configuration.

The phrase “alternative amino acid” further includes isomeric forms of the amino acid, for example, the phrase encompasses both D and L forms of amino acids.

The phrase “alternative amino acid” further includes in addition to the twenty commonly occurring amino acids that are typically found in naturally occurring polypeptides, rare amino acids, for example, canavanine, ornithine, methyl-alanine, methionine sulfoxide, and 5-hydroxytryptophane, and artificial amino acids, that is to say amino acids not normally found in vivo, for example t-butylglycine. Any chiral form of an amino acid may be used.

The phrase “alternative amino acid” further includes amino acids that are chemically modified at its amino side group. Chemical modification includes adding chemical moieties, creating new bonds, and removing chemical moieties. Modifications at amino side groups include, but are not limited to, acylation of lysine e-amino groups, N-alkylation of arginine, histidine, or lysine, alkylation of glutamic or aspartic acids carboxylic groups, and deamination of glutamine or asparagine. Modifications of the terminal amino acid include, but are not limited to, the des-amino, N-lower alkyl, N-di-lower alkyl, and N-acyl modifications. Modifications of the terminal carboxy group include, but are not limited to, the amide, lower alkyl amide, dialyl amide, and lower alkyl ester modifications. A lower alkyl is a C₃-C₄ alkyl. Further more, one or more side groups, or terminal groups, may be protected by protective groups known to the ordinary chemist. The alpha carbon of an amino acid may be mono- or di-methylated.

In particular aspects, the neuromedin U receptor agonist optionally includes a protecting group covalently joined to the N-terminal amino group. A protecting group covalently joined to the N-terminal amino group of the neuromedin U receptor agonists reduces the reactivity of the amino terminus under in vivo conditions. Amino protecting groups include —C₁₋₁₀ alkyl, —C₁₋₁₀ substituted alkyl, —C₂₋₁₀ alkenyl, —C₂₋₁₀ substituted alkenyl, aryl, —C₁₋₆ alkyl aryl, —C(O)—(CH₂)₁₋₆—COOH, —C(O)—C₁₋₆ alkyl, —C(O)-aryl, —C(O)—O—C₁₋₆ alkyl, or —C(O)—O-aryl. In particular embodiments, the amino terminus protecting group is selected from the group consisting of acetyl, propyl, succinyl, benzyl, benzyloxycarbonyl, and t-butyloxycarbonyl. Deamination of the N-terminal amino acid is another modification that is contemplated for reducing the reactivity of the amino terminus under in vivo conditions.

Chemically modified compositions of the neuromedin U receptor agonists wherein the neuromedin U receptor agonist derivatives are linked to a polymer are also included within the scope of the present invention. The polymer selected is usually modified to have a single reactive group, such as an active ester for acylation or an aldehyde for alkylation, so that the degree of polymerization may be controlled as provided for in the present methods. Included within the scope of polymers is a mixture of polymers. Preferably, for therapeutic use of the end-product preparation, the polymer will be pharmaceutically acceptable.

The polymer or mixture thereof may be selected from the group consisting of; for example, polyethylene glycol (PEG), monomethoxy-polyethylene glycol, dextran, cellulose, or other carbohydrate based polymers, poly-(N-vinyl pyrrolidone) polyethylene glycol, propylene glycol homopolymers, a polypropylene oxide/ethylene oxide co-polymer, polyoxyethylated polyols (for example, glycerol), and polyvinyl alcohol.

In further still embodiments, the neuromedin U receptor agonists are modified by PEGylation, cholesterylation, or palmitoylation. The modification can be to any amino acid residue in the neuromedin U receptor agonist, however, in currently embodiments, the modification is to the N-terminal amino acid of the neuromedin U receptor agonist, either directly to the N-terminal amino acid or by way coupling to the thiol group of a cysteine residue added to the N-terminus or a linker added to the N-terminus such as Ttds. In further embodiments, the N-terminus of the neuromedin U receptor agonist comprises a cysteine residue to which a protecting group is coupled to the N-terminal amino group of the cysteine residue and the cysteine thiolate group is derivatized with N-ethylmaleimide, PEG group, cholesterol group, or palmitoyl group. In further still embodiments, an acetylated cysteine residue is added to the N-terminus of the neuromedin U receptor agonists, and the thiol group of the cysteine is derivatized with N-ethylmaleimide, PEG group, cholesterol group, or palmitoyl group.

It is well known that the properties of certain proteins can be modulated by attachment of polyethylene glycol (PEG) polymers, which increases the hydrodynamic volume of the protein and thereby slows its clearance by kidney filtration. (See, for example, Clark et al., J. Biol. Chem. 271: 21969-21977 (1996)). Therefore, it is envisioned that the core peptide residues can be PEGylated to provide enhanced therapeutic benefits such as, for example, increased efficacy by extending half-life in vivo. Thus, PEGylating the neuromedin U receptor agonists will improve the pharmacokinetics and pharmacodynamics of the neuromedin U receptor agonists.

Peptide PEGylation methods are well known in the literature and described in the following references, each of which is incorporated herein by reference: Lu et al., Int. J. Pept. Protein Res.43: 127-38 (1994); Lu et al., Pept. Res. 6: 140-6 (1993); Felix et al., Int. J. Pept. Protein Res. 46: 253-64 (1995); Gaertner et al., Bioconjug. Chem. 7: 38-44 (1996); Tsutsumi et al., Thromb. Haemost. 77: 168-73 (1997); Francis et al., hit. J. Hematol. 68: 1-18 (1998); Roberts et al., J. Pharm. Sci. 87: 1440-45 (1998); and Tan et al., Protein Expr. Purif. 12: 45-52 (1998). Polyethylene glycol or PEG is meant to encompass any of the forms of PEG that have been used to derivatize other proteins, including, but not limited to, mono-(C₁₋₁₀) alkoxy or aryloxy-polyethylene glycol. Suitable PEG moieties include, for example, 40 kDa methoxy poly(ethylene glycol) propionaldehyde (Dow, Midland, Mich.); 60 kDa methoxy poly(ethylene glycol) propionaldehyde (Dow, Midland, Mich.); 40 kDa methoxy poly(ethylene glycol) maleimido-propionamide (Dow, Midland, Mich.); 31 kDa alpha-methyl-w-(3-oxopropoxy), polyoxyethylene (NOF Corporation, Tokyo); mPEG₂-NHS-40k (Nektar); mPEG₂-MAL-40k (Nektar), SUNBRIGHT GL2-400MA ((PEG)₂40kDa) (NOF Corporation, Tokyo), SUNBRIGHT ME-200MA (PEG20kDa) (NOF Corporation, Tokyo). The PEG groups are generally attached to the neuromedin U receptor agonists via acylation or alkylation through a reactive group on the PEG moiety (for example, a maleimide, an aldehyde, amino, thiol, or ester group) to a reactive group on the neuromedin U receptor agonist (for example, an aldehyde, amino, thiol, a maleimide, or ester group).

The PEG molecule(s) may be covalently attached to any Lys, Cys, or K(CO(CH₂)₂SH) residues at any position in the neuromedin U receptor agonist. The neuromedin U receptor agonists described herein can be PEGylated directly to any amino acid at the N-terminus by way of the N-terminal amino group. A “linker arm” may be added to the neuromedin U receptor agonist to facilitate PEGylation. PEGylation at the thiol side-chain of cysteine has been widely reported (See, e.g., Caliceti & Veronese, Adv. Drug Deliv. Rev. 55: 1261-77 (2003)). If there is no cysteine residue in the peptide, a cysteine residue can be introduced through substitution or by adding a cysteine to the N-terminal amino acid. Those neuromedin U receptor agonists, which have been PEGylated, have been PEGylated through the side chains of a cysteine residue added to the N-terminal amino acid.

In some aspects, the PEG molecule(s) may be covalently attached to an amide group in the C-terminus of the neuromedin U receptor agonist. In general, there is at least one PEG molecule covalently attached to the neuromedin U receptor agonist. In particular aspects, the PEG molecule is branched while in other aspects, the PEG molecule may be linear. In particular aspects, the PEG molecule is between 1 kDa and 100 kDa in molecular weight. In further aspects, the PEG molecule is selected from 10, 20, 30, 40, 50, 60, and 80 kDa. In further still aspects, it is selected from 20, 40, or 60 kDa. Where there are two PEG molecules covalently attached to the neuromedin U receptor agonist of the present invention, each is 1 to 40 kDa and in particular aspects, they have molecular weights of 20 and 20 kDa, 10 and 30 kDa, 30 and 30 kDa, 20 and 40 kDa, or 40 and 40 kDa. In particular aspects, the neuromedin U receptor agonists contain mPEG-cysteine. The mPEG in mPEG-cysteine can have various molecular weights. The range of the molecular weight is preferably 5 kDa to 200 kDa, more preferably 5 kDa to 100 kDa, and further preferably 20 kDa to 60 kDA. The mPEG can be linear or branched.

Currently, it is preferable that the neuromedin U receptor agonists are PEGylated through the side chains of a cysteine added to the N-terminal amino acid. Currently, the agonists preferably contain mPEG-cysteine. The mPEG in mPEG-cysteine can have various molecular weights. The range of the molecular weight is preferably 5 kDa to 200 kDa, more preferably 5 kDa to 100 kDa, and further preferably 20 kDa to 60 kDA. The mPEG can be linear or branched.

A useful strategy for the PEGylation of synthetic neuromedin U receptor agonists consists of combining, through forming a conjugate linkage in solution, a peptide, and a PEG moiety, each bearing a special functionality that is mutually reactive toward the other. The neuromedin U receptor agonists can be easily prepared with conventional solid phase synthesis. The neuromedin U receptor agonist is “preactivated” with an appropriate functional group at a specific site. The precursors are purified and fully characterized prior to reacting with the PEG moiety. Conjugation of the peptide with PEG usually takes place in aqueous phase and can be easily monitored by reverse phase analytical HPLC. The PEGylated neuromedin U receptor agonist can be easily purified by cation exchange chromatography or preparative HPLC and characterized by analytical HPLC, amino acid analysis and laser desorption mass spectrometry.

The neuromedin U receptor agonist can comprise other non-sequence modifications, for example, glycosylation, lipidation, acetylation, phosphorylation, carboxylation, methylation, or any other manipulation or modification, such as conjugation with a labeling component. While, in particular aspects, the neuromedin U receptor agonist herein utilize naturally-occurring amino acids or D isoforms of naturally occurring amino acids, substitutions with non-naturally occurring amino acids (for example., methionine sulfoxide, methionine methylsulfonium, norleucine, epsilon-aminocaproic acid, 4-aminobutanoic acid, tetrahydroisoquinoline-3-carboxylic acid, 8-aminocaprylic acid, 4 aminobutyric acid, Lys(N(epsilon)-trifluoroacetyl) or synthetic analogs, for example, o-aminoisobutyric acid, p or y-amino acids, and cyclic analogs. In further still aspects, the neuromedin U receptor agonists comprise a fusion protein that having a first moiety, which is a neuromedin U receptor agonist, and a second moiety, which is a heterologous peptide.

The neuromedin U receptor agonist may be modified by a variety of chemical techniques to produce derivatives having essentially the same activity as the unmodified neuromedin U receptor agonist and/or having other desirable properties. A protecting group covalently joined to the C-terminal carboxy group reduces the reactivity of the carboxy terminus under in vivo conditions. For example, carboxylic acid groups of the peptide, whether carboxyl-terminal or side chain, may be provided in the form of a salt of a pharmacologically-acceptable cation or esterified to form a C₁₋₆ ester, or converted to an amide of formula NRR₂ wherein R and R₂ are each independently H or C₁₋₆ alkyl, or combined to form a heterocyclic ring, such as a 5- or 6-membered ring. The carboxy terminus protecting group is preferably attached to the α-carbonyl group of the last amino acid. Carboxy terminus protecting groups include, but are not limited to, amide, methylamide, and ethylamide. Amino groups of the peptide, whether N-terminal or side chain, may be in the form of a pharmacologically-acceptable acid addition salt, such as the HCl, HBr, acetic, benzoic, toluene sulfonic, maleic, tartaric, and other organic salts, or may be modified to C₁₋₆ alkyl or dialkyl amino or further converted to an amide.

Hydroxyl groups of the neuromedin U receptor agonist side chain may be converted to C₁₋₆ alkoxy or to a C₁₋₆ ester using well-recognized techniques. Phenyl and phenolic rings of the peptide side chain may be substituted with one or more halogen atoms, such as fluorine, chlorine, bromine or iodine, or with C₁₋₆ alkyl, C₁₋₆ alkoxy, carboxylic acids and esters thereof, or amides of such carboxylic acids. Methylene groups of the neuromedin U receptor agonist side chains can be extended to homologous C₂₋₄ alkylenes. Thiols can be protected with any one of a number of well-recognized protecting groups, such as acetamide groups. Those skilled in the art will also recognize methods for introducing cyclic structures into the peptides of this invention to select and provide conformational constraints to the structure that result in enhanced stability. For example, a carboxyl-terminal or amino-terminal cysteine residue can be added to the peptide, so that when oxidized the peptide will contain a disulfide bond, thereby generating a cyclic peptide. Other peptide cyclizing methods include the formation of thioethers and carboxyl- and amino-terminal amides and esters. Another method to provide conformational constraints to the structure that result in enhanced stability relies on the substitution of one or more amino acids with N-alkyl-amino acids.

Polysaccharide polymers are another type of water soluble polymer that may be used for protein modification. Dextrans are polysaccharide polymers comprised of individual subunits of glucose predominantly linked by α1-6 linkages. The dextran itself is available in many molecular weight ranges, and is readily available in molecular weights from about 1 kDa to about 70 kDa. Dextran is a suitable water soluble polymer for use as a vehicle by itself or in combination with another vehicle (See, for example, WO96/11953 and WO96/05309). The use of dextran conjugated to therapeutic or diagnostic immunoglobulins has been reported; see, for example, European Patent Publication No. 0 315 456. Dextran of about 1 kDa to about 20 kDa is preferred when dextran is used as a vehicle in accordance with the present invention.

As described above, the presence of a “linker” group is optional. When present, its chemical structure is not critical, since it serves primarily as a spacer. However, in certain embodiments, the linker may itself provide improved properties to the compositions of the present invention. The linker is preferably made up of amino acids linked together by peptide bonds. Thus, in particular embodiments, the linker is made up of from 1 to 20 amino acids linked by peptide bonds, wherein the amino acids are selected from the 20 naturally occurring amino acids. Some of these amino acids may be glycosylated, as is well understood by those in the art. In a further embodiment, the 1 to 20 amino acids are selected from glycine, alanine, proline, asparagine, glutamine, and lysine. Even more preferably, a linker is made up of a majority of amino acids that are sterically unhindered, such as glycine and alanine. Thus, linkers include are polyglycines (particularly (Gly)₄, (Gly)₅), poly(Gly-Ala), and polyalanines. Other specific examples of linkers are (Gly)₃Lys(Gly)₄; (Gly)₃AsnGlySer(Gly)₂; (Gly)₃Cys(Gly)₄; and GlyProAsnGlyGly.

Non-peptide linkers can also be used. For example, alkyl linkers such as-NH—(CH₂)ₛ—C(O)—, wherein s=2-20 could be used. These alkyl linkers may further be substituted by any non-sterically hindering group such as lower alkyl (for example, C₁₋₆) lower acyl, halogen (for example, Cl, Br), CN, NH₂, phenyl, and the like. An exemplary non-peptide linker is a PEG linker, wherein n is such that the linker has a molecular weight of 100 to 5000 kD, preferably 100 to 500 kD. The peptide linkers may be altered to form derivatives in the same manner as described above. Other linkers include Ttds (N-(13-amino-4,7,10-trioxa-tridecayl)-succinamic acid).

The present invention includes diastereomers as well as their racemic and resolved enantiomerically pure forms. The neuromedin U receptor agonists can contain D-amino acids, L-amino acids, or a combination thereof. In general, the amino acids are in the L-form with particular amino acids in D-form. As is known in the art, individual amino acids can be represented as follows: A=Ala=Alanine; C=Cys=Cysteine; D=Asp=Aspartic Acid; E=Glu=Glutamic Acid; F=Phe=Phenylalanine; G=Gly=Glycine; H=His=Histidine; I=Ile=Isoleucine; K=Lys=Lysine; L=Leu=Leucine; M=Met=Methionine; N=Asn=Asparagine; P=Pro=Proline; Q=Gln=Glutamine; R=Arg=Arginine; S=Ser=Serine; T=Thr=Threonine; V=Val=Valine; W=Trp=Tryptophan; and Y=Tyr=Tyrosine.

The neuromedinU receptor agonists can be linked, conjugated, or fused to a carrier molecule such as albumin, transferrrin, or an antibody or antibody fragment such as the Fab or Fc fragment. For example, the neuromedin U receptor agonists can be linked via a linker moiety to a catalytic antibody such as disclosed in U.S. Pub. Application Nos. US20030175921, US200301960676, and US20030129188, which describes linking of various peptides to the catalytic site of an aldolase catalytic antibody. The neuromedin U receptor agonists can be conjugated to an Fc fragment via linker moiety or fused to the Fc fragment of an antibody in the form of a fusion protein such as disclosed for the GLP-1-Fc fusion proteins in International Applications WO2002/046227 and WO2005/007809. The neuromedin U receptor agonist can be linked via a linker moiety to serum albumin, similar to the linking of GLP-1 to albumin as disclosed in International application WO2000/069911 or U.S. Pub application US20070093417. The neuromedin U receptor agonist can fused to a carrier molecule such as transferrin, for example similar to the transferrin-GLP-1 fusion proteins disclosed in U.S. Pat. No. 7,176,278 or albumin-GLP-1 fusion proteins described in U.S. Pat. No. 7,141,547.

Examples of neuromedin U receptor agonists of the present invention comprising the amino acid sequence ILQRGSGTAAVDFTKKDHTATWGRPFFLFRPRN (SEQ ID NO:1) are shown in Table 1. The neuromedin U receptor agonists are protected at the C-terminus with an amino group and at the N-terminus with an acetyl group (except for neuromedin U receptor agonist NMS). With the exception of neuromedin U receptor agonist NMS and NMS5, the neuromedin U receptor agonists further include a cysteine residue at the N-terminus to which an acetyl group is covalently linked to the amino group of the cysteine residue. As shown in the table, for many of the neuromedin U receptor agonists, the thiol group of the cysteine residue is reacted with a second group. For example, for neuromedin U receptor agonists in the table shown with a C₁ at the N-terminus, the neuromedin U receptor agonist has an N-acetylated cysteine residue at the N-terminus of the neuromedin U receptor agonist linked by way of its thiol group to N-ethylmaleimidyl; for neuromedin U receptor agonists in the table shown with a C₂ at the N-terminus, the neuromedin U receptor agonist has an N-acetylated cysteine residue at the N-terminus of the neuromedin U receptor agonist linked by way of its thiol group to a branched (PEG)₂40kDa; for neuromedin U receptor agonists shown with a C₄ at the N-terminus of the neuromedin U receptor agonist, the neuromedin U receptor agonist has an N-acetylated cysteine residue at the N-terminus of the neuromedin U receptor agonist linked by way of its thiol group to a linear (PEG)40kDa; for neuromedin U receptor agonists shown with a C₃ at the N-terminus of the neuromedin U receptor agonist, the neuromedin U receptor agonist has an N-acetylated cysteine residue at the N-terminus of the neuromedin U receptor agonist linked by way of its thiol group to Cholesterol.

TABLE 1

| SEQ | | |
|---|---|---|
| ID | | |
| NO. | Peptides | Neuromedin U Receptor Agonist Sequences |
| 1 | NMS | ILQRGSGTAAVDFTKKDHTATWGRPFFLFRPRN-CONH₂ |
| 2 | NMS′ | Ac-CILQRGSGTAAVDFTKKDHTATWGRPFFLFRPRN-CONH₂ |
| 2 | NMS3 | Ac-C₁ILQRGSGTAAVDFTKKDHTATWGRPFFLFRPRN-CONH₂ |
| 2 | NMS1 | Ac-C₂ILQRGSGTAAVDFTKKDHTATWGRPFFLFRPRN-CONH₂ |
| 2 | NMS2 | Ac-C₃ILQRGSGTAAVDFTKKDHTATWGRPFFLFRPRN-CONH₂ |
| 2 | NMS4 | Ac-C₄ILQRGSGTAAVDFTKKDHTATWGRPFFLFRPRN-CONH₂ |
| 1 | NMS5 | Pam-ILQRGSGTAAVDFTKKDHTATWGRPFFLFRPRN-CONH₂ |

C = cysteine; C₁ = Cys(N-ethylmaleimidyl), C₂ = Cys(PEG)₂40 kDa, C₄ = Cys(PEG)40 kDa; each corresponding to a cysteine residue PEGylated via the side-chain thiol with a branched PEG [(PEG)₂] or a linear PEG of the indicated MW; C₃ = Cys(Cholesteroyl), corresponding to a cysteine residue linked to cholesterol via the side-chain thiol; Ac = acetyl; Pam =
palmitoyl

The Neuromedin U receptor agonists shown in Table 1 were designed starting from the native human Neuromedin S peptide (SEQ ID NO:1, NMS) and adding an acetylated cysteine residue at the N-terminus to make the analog, NMS′. The cysteine thiolated group of NMS′ was derivatized with (a) N-ethylmaleimide to obtain the analog, NMS3, that is a control peptide; (b) (PEG)₂40kDa-maleimide to obtain the analog, NMS1, this PEGylated analog was designed to improve the pharmacological profile in vivo; (c) a bromo-cholesterol group to obtain NMS2, this lipidated analog was designed to improve the pharmacological profile; or (d) (PEG)40kDa-maleimide to obtain analog, NMS4, this PEGylated analog was designed to improve the pharmacological profile in vivo.

The analog, NMS5, was based on the native human Neuromedin S in which the N-terminal amino group was capped with palmitic acid. It was designed to study the effect of N-terminal palmitoylation on efficacy, or more generally, the effect of acylation with a fatty acid chain on efficacy.

The sites of PEGylation on the Neuromedin U receptor agonists shown in Table 1 were chosen taking into account the structure of NMS and its interactions with its receptors. Hence, the PEGylation is preferably site-specific. PEGylation at the thiol side-chain of cysteine has been widely reported (See, e.g., Caliceti & Veronese, Adv. Drug Deliv. Rev. 55: 1261-77 (2003)). If there is no cysteine residue in the peptide, a cysteine residue can be introduced through substitution or by adding a cysteine to the N-terminal amino acid. Those neuromedin U receptor agonists, which have been PEGylated, have been PEGylated through the side chains of a cysteine residue added to the N-terminal amino acid. As discussed supra, the PEG in Cys(PEG)teine can have various molecular weights. The range of the molecular weight is preferably 5 kDa to 200 kDa, more preferably 5 kDa to 100 kDa, and further preferably 20 kDa to 40 kDA. The PEG can be linear or branched.

### Pharmaceutical Compositions

Further provided are pharmaceutical compositions comprising a therapeutically effective amount of one or more of the neuromedin U receptor agonists disclosed herein for the treatment of a metabolic disorder in an individual. Such disorders include, but are not limited to, obesity, metabolic syndrome or syndrome X, type II diabetes, complications of diabetes such as retinopathy, hypertension, dyslipidemias, cardiovascular disease, gallstones, osteoarthritis, and certain forms of cancers. The obesity-related disorders herein are associated with, caused by, or result from obesity.

“Obesity” is a condition in which there is an excess of body fat. The operational definition of obesity is based on the Body Mass Index (BMI), calculated as body weight per height in meters squared (kg/m2). “Obesity” refers to a condition whereby an otherwise healthy subject has a Body Mass Index (BMI) greater than or equal to 30 kg/m2, or a condition whereby a subject with at least one co-morbidity has a BMI greater than or equal to 27 kg/m2. An “obese subject” is an otherwise healthy subject with a Body Mass Index (BMI) greater than or equal to 30 kg/m2 or a subject with at least one co-morbidity with a BMI greater than or equal to 27 kg/m2. A “subject at risk for obesity” is an otherwise healthy subject with a BMI of 25 kg/m2 to less than 30 kg/m2 or a subject with at least one co-morbidity with a BMI of 25 kg/m2 to less than 27 kg/m2.

The increased risks associated with obesity occur at a lower Body Mass Index (BMI) in Asians. In Asian countries, including Japan, “obesity” refers to a condition whereby a subject with at least one obesity-induced or obesity-related co-morbidity that requires weight reduction or that would be improved by weight reduction, has a BMI greater than or equal to 25 kg/m2. In Asian countries, including Japan, an “obese subject” refers to a subject with at least one obesity-induced or obesity-related co-morbidity that requires weight reduction or that would be improved by weight reduction, with a BMI greater than or equal to 25 kg/m2. In Asian countries, a “subject at risk of obesity” is a subject with a BMI of greater than 23 kg/m2 to less than 25 kg/m2.

As used herein, the term “obesity” is meant to encompass all of the above definitions of obesity.

Obesity-induced or obesity-related co-morbidities include, but are not limited to, diabetes, non-insulin dependent diabetes mellitus—type 2, impaired glucose tolerance, impaired fasting glucose, insulin resistance syndrome, dyslipidemia, hypertension, hyperuricacidemia, gout, coronary artery disease, myocardial infarction, angina pectoris, sleep apnea syndrome, Pickwickian syndrome, fatty liver; cerebral infarction, cerebral thrombosis, transient ischemic attack, orthopedic disorders, arthritis deformans, lumbodynia, emmeniopathy, and infertility. In particular, co-morbidities include: hypertension, hyperlipidemia, dyslipidemia, glucose intolerance, cardiovascular disease, sleep apnea, diabetes mellitus, and other obesity-related conditions.

“Treatment” (of obesity and obesity-related disorders) refers to the administration of the compounds of the present invention to reduce or maintain the body weight of an obese subject. One outcome of treatment may be reducing the body weight of an obese subject relative to that subject's body weight immediately before the administration of the compounds of the present invention. Another outcome of treatment may be preventing body weight regain of body weight previously lost as a result of diet, exercise, or pharmacotherapy. Another outcome of treatment may be decreasing the occurrence of and/or the severity of obesity-related diseases. The treatment may suitably result in a reduction in food or calorie intake by the subject, including a reduction in total food intake, or a reduction of intake of specific components of the diet such as carbohydrates or fats; and/or the inhibition of nutrient absorption; and/or the inhibition of the reduction of metabolic rate; and in weight reduction in patients in need thereof. The treatment may also result in an alteration of metabolic rate, such as an increase in metabolic rate, rather than or in addition to an inhibition of the reduction of metabolic rate; and/or in minimization of the metabolic resistance that normally results from weight loss.

“Prevention” (of obesity and obesity-related disorders) refers to the administration of the compounds of the present invention to reduce or maintain the body weight of a subject at risk of obesity. One outcome of prevention may be reducing the body weight of a subject at risk of obesity relative to that subject's body weight immediately before the administration of the compounds of the present invention. Another outcome of prevention may be preventing body weight regain of body weight previously lost as a result of diet, exercise, or pharmacotherapy. Another outcome of prevention may be preventing obesity from occurring if the treatment is administered prior to the onset of obesity in a subject at risk of obesity. Another outcome of prevention may be decreasing the occurrence and/or severity of obesity-related disorders if the treatment is administered prior to the onset of obesity in a subject at risk of obesity. Moreover, if treatment is commenced in already obese subjects, such treatment may prevent the occurrence, progression or severity of obesity-related disorders, such as, but not limited to, arteriosclerosis, Type II diabetes, polycystic ovarian disease, cardiovascular diseases, osteoarthritis, dermatological disorders, hypertension, insulin resistance, hypercholesterolemia, hypertriglyceridemia, and cholelithiasis.

The obesity-related disorders herein are associated with, caused by, or result from obesity. Examples of obesity-related disorders include overeating and bulimia, hypertension, diabetes, elevated plasma insulin concentrations and insulin resistance, dyslipidemias, hyperlipidemia, endometrial, breast, prostate and colon cancer, osteoarthritis, obstructive sleep apnea, cholelithiasis, gallstones, heart disease, abnormal heart rhythms and arrythmias, myocardial infarction, congestive heart failure, coronary heart disease, sudden death, stroke, polycystic ovarian disease, craniopharyngioma, the Prader-Willi Syndrome, Frohlich's syndrome, GH-deficient subjects, normal variant short stature, Turner's syndrome, and other pathological conditions showing reduced metabolic activity or a decrease in resting energy expenditure as a percentage of total fat-free mass, e.g., children with acute lymphoblastic leukemia. Further examples of obesity-related disorders are metabolic syndrome, also known as syndrome X, insulin resistance syndrome, sexual and reproductive dysfunction, such as infertility, hypogonadism in males and hirsutism in females, gastrointestinal motility disorders, such as obesity-related gastro-esophageal reflux, respiratory disorders, such as obesity-hypoventilation syndrome (Pickwickian syndrome), cardiovascular disorders, inflammation, such as systemic inflammation of the vasculature, arteriosclerosis, hypercholesterolemia, hyperuricaemia, lower back pain, gallbladder disease, gout, and kidney cancer. The compounds of the present invention are also useful for reducing the risk of secondary outcomes of obesity, such as reducing the risk of left ventricular hypertrophy.

The term “diabetes,” as used herein, includes both insulin-dependent diabetes mellitus (IDDM, also known as type I diabetes) and non-insulin-dependent diabetes mellitus (NIDDM, also known as Type II diabetes). Type I diabetes, or insulin-dependent diabetes, is the result of an absolute deficiency of insulin, the hormone which regulates glucose utilization. Type II diabetes, or insulin-independent diabetes (i.e., non-insulin-dependent diabetes mellitus), often occurs in the face of normal, or even elevated levels of insulin and appears to be the result of the inability of tissues to respond appropriately to insulin. Most of the Type II diabetics are also obese. The compounds of the present invention are useful for treating both Type I and Type II diabetes. The compounds are especially effective for treating Type II diabetes. The compounds of the present invention are also useful for treating and/or preventing gestational diabetes mellitus.

The neuromedin U receptor agonists disclosed herein may be used in a pharmaceutical composition when combined with a pharmaceutically acceptable carrier. Such compositions comprise a therapeutically-effective amount of the neuromedin U receptor agonist and a pharmaceutically acceptable carrier. Such a composition may also be comprised of (in addition to neuromedin U receptor agonist and a carrier) diluents, fillers, salts, buffers, stabilizers, solubilizers, and other materials well known in the art. Compositions comprising the neuromedin U receptor agonists can be administered, if desired, in the form of salts provided the salts are pharmaceutically acceptable. Salts may be prepared using standard procedures known to those skilled in the art of synthetic organic chemistry.

The term “individual” is meant to include humans and companion or domesticated animals such as dogs, cats, horses, and the like. Therefore, the compositions comprising formula I are also useful for treating or preventing obesity and obesity-related disorders in cats and dogs. As such, the term “mammal” includes companion animals such as cats and dogs.

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. The term “pharmaceutically acceptable salt” further includes all acceptable salts such as acetate, lactobionate, benzenesulfonate, laurate, benzoate, malate, bicarbonate, maleate, bisulfate, mandelate, bitartrate, mesylate, borate, methylbromide, bromide, methylnitrate, calcium edetate, methylsulfate, camsylate, mucate, carbonate, napsylate, chloride, nitrate, clavulanate, N-methylglucamine, citrate, ammonium salt, dihydrochloride, oleate, edetate, oxalate, edisylate, pamoate (embonate), estolate, palmitate, esylate, pantothenate, fumarate, phosphate/diphosphate, gluceptate, polygalacturonate, gluconate, salicylate, glutamate, stearate, glycollylarsanilate, sulfate, hexylresorcinate, subacetate, hydrabamine, succinate, hydrobromide, tannate, hydrochloride, tartrate, hydroxpiaphthoate, teoclate, iodide, tosylate, isothionate, triethiodide, lactate, panoate, valerate, and the like which can be used as a dosage form for modifying the solubility or hydrolysis characteristics or can be used in sustained release or pro-drug formulations. It will be understood that, as used herein, references to the neuromedin U receptor agonists of the general formula (I) are meant to also include the pharmaceutically acceptable salts.

As utilized herein, the term “pharmaceutically acceptable” means a non-toxic material that does not interfere with the effectiveness of the biological activity of the active ingredient(s), approved by a regulatory agency of the Federal or a state government or listed in the U.S. Pharmacopoeia or other generally recognized pharmacopoeia for use in animals and, more particularly, in humans. The term “carrier” refers to a diluent, adjuvant, excipient, or vehicle with which the therapeutic is administered and includes, but is not limited to such sterile liquids as water and oils. The characteristics of the carrier will depend on the route of administration. The neuromedin U receptor agonist may be in multimers (for example, heterodimers or homodimers) or complexes with itself or other peptides. As a result, pharmaceutical compositions of the invention may comprise one or more neuromedin U receptor agonists in such multimeric or complexed form.

As used herein, the term “therapeutically effective amount” means the total amount of each active component of the pharmaceutical composition or method that is sufficient to show a meaningful patient benefit, i.e., treatment, healing, prevention or amelioration of the relevant medical condition, or an increase in rate of treatment, healing, prevention or amelioration of such conditions. When applied to an individual active ingredient, administered alone, the term refers to that ingredient alone. When applied to a combination, the term refers to combined amounts of the active ingredients that result in the therapeutic effect, whether administered in combination, serially, or simultaneously.

The pharmacological composition can comprise one or more neuromedin U receptor agonists; one or more neuromedin U receptor agonists and one or more other agents for treating a metabolic disorder; or the pharmacological composition comprising the one or more neuromedin U receptor agonists can be used concurrently with a pharmacological composition comprising an agent for treating a metabolic disorder. Such disorders include, but are not limited to, obesity, metabolic syndrome or syndrome X, type II diabetes, complications of diabetes, hypertension, dyslipidemias, cardiovascular disease, gallstones, osteoarthritis, and certain forms of cancers.

When the pharmacological composition comprises another agent for treating a metabolic disorder or the treatment includes a second pharmacological composition comprising an agent for treating a metabolic disorder, the agent includes, but are not limited to, cannabinoid (CB1) receptor antagonists, glucagon like peptide 1 (GLP-1) receptor agonists, lipase inhibitors, leptin, tetrahydrolipstatin, 2-4-dinitrophenol, acarbose, sibutramine, phentamine, fat absorption blockers, simvastatin, mevastatin, ezetimibe, atorvastatin, sitagliptin, metformin, orlistat, Qnexa, topiramate, naltrexone, bupriopion, phentermine, losartan, losartan with hydrochlorothiazide, and the like.

Suitable agents of use in combination with a compound of the present invention, include, but are not limited to:

(a) NMU derivative and analogs disclosed in commonly owned International Application No. PCT/US2007/006635 and comprising the general formula (II)

Z¹-peptide-Z²

wherein the peptide has the amino acid sequence X¹—X²—X³—X⁴—X⁵—X⁶—X⁷—X⁸—X⁹—X¹⁰—X¹¹—X¹²—X¹³—X¹⁴—X¹⁵—X¹⁶—X¹⁷—X¹⁸—X¹⁹—X²⁰—X²¹—X²²—X²³—X²⁴—X²⁵ (SEQ ID NO:4) wherein amino acids 1 to 17 can be any amino acid or absent, wherein amino acid X¹⁸ is absent, Y, W, F, a des-amino acid or an acyl group; amino acid X¹⁹ is A, W, Y, F or an aliphatic amino acid; amino acid X²⁰ is absent, L, G, sarcosine (Sar), D-Leu, NMe-Leu, D-Ala or A; amino acid X²¹ is F, NMe-Phe, an aliphatic amino acid, an aromatic amino acid, A or W; X²² is R, K, A or L; amino acid X²³ is P, Sar, A or L; amino acid X²⁴ is R, Harg or K; and amino acid X²⁵ is N, any D- or L-amino acid, Nle or D-Nle, A; and Z¹ is an optionally present protecting group that, if present, is joined to the N-terminal amino group; and Z² is NH₂ or an optionally present protecting group that, if present, is joined to the C-terminal carboxy group, and pharmaceutically acceptable salts thereof.

In particular embodiments, the peptide comprises the amino acid sequence X¹—X²—X³—X⁴—X⁵—X⁶—X⁷—X⁸—X⁹—X¹⁰—X¹¹—X¹²—X¹³—X¹⁴—X¹⁵—X¹⁶—X¹⁷—X¹⁸—F-L-F—R—P—R—N (SEQ ID NO:5) wherein amino acids 1 to 17 can be any amino acid or absent.

In further embodiments, the peptide comprises the amino acid sequence F—R—V-D-E-E-F-Q-S—P—F-A-S-Q-S—R-G-X¹⁸—X¹⁹—X²⁰—X²¹—X²²—X²³—X²⁴—X²⁵ (SEQ ID NO:6) wherein amino acid X¹⁸ is absent, Y, W, F, a des-amino acid or an acyl group; amino acid X¹⁹ is A, W, Y, F or an aliphatic amino acid; amino acid X²⁰ is absent, G, sarcosine (Sar), D-Leu, NMe-Leu, D-Ala or A; amino acid X²¹ is NMe-Phe, an aliphatic amino acid, an aromatic amino acid, A or W; amino acid X²² is K, A or L; amino acid X²³ is Sar, A or L; amino acid X²⁴ is Harg or K; and amino acid X²⁵ is any D- or L-amino acid, Nle or D-Nle, or A.

In yet another embodiment, the peptide comprises the amino acid sequence X¹—X²—X³—X⁴—X⁵—X⁶—X⁷—X⁸ (SEQ ID NO:7) wherein amino acid X¹ is absent, Y, W, F, a des-amino acid or an acyl group; amino acid X² is A, W, Y, F or an aliphatic amino acid; amino acid X³ is absent, G, sarcosine (Sar), D-Leu, NMe-Leu, D-Ala or A; amino acid X⁴ is NMe-Phe, an aliphatic amino acid, an aromatic amino acid, A or W; amino acid X⁵ is K, A or L; amino acid X⁶ is Sar, A or L; amino acid X⁷ is Harg or K; and amino acid X⁸ is any D- or L-amino acid, Nle or D-Nle, or A.
(b) anti-diabetic agents such as (1) PPARγ agonists such as glitazones (e.g. ciglitazone; darglitazone; englitazone; isaglitazone (MCC-555); pioglitazone (ACTOS); rosiglitazone (AVANDIA); troglitazone; rivoglitazone, BRL49653; CLX-0921; 5-BTZD, GW-0207, LG-100641, R483, and LY-300512, and the like and compounds disclosed in WO97/10813, 97/27857, 97/28115, 97/28137, 97/27847, 03/000685, and 03/027112 and SPPARMS (selective PPAR gamma modulators) such as T131 (Amgen), FK614 (Fujisawa), netoglitazone, and metaglidasen; (2) biguanides such as buformin; metformin; and phenformin, and the like; (3) protein tyrosine phosphatase-1B (PTP-1B) inhibitors such as ISIS 113715, A-401674, A-364504, IDD-3, IDD 2846, KP-40046, 1CR61639, MC52445, MC52453, C7, OC-060062, OC-86839, OC29796, TTP-277BC1, and those agents disclosed in WO 04/041799, 04/050646, 02/26707, 02/26743, 04/092146, 03/048140, 04/089918, 03/002569, 04/065387, 04/127570, and US 2004/167183; (4) sulfonylureas such as acetohexamide; chlorpropamide; diabinese; glibenclamide; glipizide; glyburide; glimepiride; gliclazide; glipentide; gliquidone; glisolamide; tolazamide; and tolbutamide, and the like; (5) meglitinides such as repaglinide, metiglinide (GLUFAST) and nateglinide, and the like; (6) alpha glucoside hydrolase inhibitors such as acarbose; adiposine; camiglibose; emiglitate; miglitol; voglibose; pradimicin-Q; salbostatin; C103-711; MDL-25,637; MDL-73,945; and MOR14, and the like; (7) alpha-amylase inhibitors such as tendamistat, trestatin, and Al-3688, and the like; (8) insulin secreatagogues such as linogliride nateglinide, mitiglinide (GLUFAST), ID1101A-4166, and the like; (9) fatty acid oxidation inhibitors, such as clomoxir, and etomoxir, and the like; (10) A2 antagonists, such as midaglizole; isaglidole; deriglidole; idazoxan; earoxan; and fluparoxan, and the like; (1H) insulin or insulin mimetics, such as biota, LP-100, novarapid, insulin detemir, insulin lispro, insulin glargine, insulin zinc suspension (lente and ultralente); Lys-Pro insulin, GLP-1 (17-36), GLP-1 (73-7) (insulintropin); GLP-1 (7-36)-NH2) exenatide/Exendin-4, Exenatide LAR, Linaglutide, AVE0010, CJC 1131, BIM51077, CS 872, TH0318, BAY-694326, GP010, ALBUGON (GLP-1 fused to albumin), HGX-007 (Epac agonist), S-23521, and compounds disclosed in WO 04/022004, WO 04/37859, and the like; (12) non-thiazolidinediones such as JT-501, and farglitazar (GW-2570/GI-262579), and the like; (13) PPARα/γ dual agonists such as AVE 0847, CLX-0940, GW-1536, GW1929, GW-2433, KRP-297, L-796449, LBM 642, LR-90, LY510919, MK-0767, ONO 5129, SB 219994, TAK-559, TAK-654, 677954 (GlaxoSmithkline), E-3030 (Eisai), LY510929 (Lilly), AK109 (Asahi), DRF2655 (Dr. Reddy), DRF8351 (Dr. Reddy), MC3002 (Maxocore), TY51501 (ToaEiyo), naveglitazar, muraglitizar, peliglitazar, tesaglitazar (GALIDA), reglitazar (JTT-501), chiglitazar, and those disclosed in WO 99/16758, WO 99/19313, WO 99/20614, WO 99/38850, WO 00/23415, WO 00/23417, WO 00/23445, WO 00/50414, WO 01/00579, WO 01/79150, WO 02/062799, WO 03/033481, WO 03/033450, WO 03/033453; and (14) other insulin sensitizing drugs; (15) VPAC2 receptor agonists; (16) GLK modulators, such as PSN105, RO 281675, RO 274375 and those disclosed in WO 03/015774, WO 03/000262, WO 03/055482, WO 04/046139, WO 04/045614, WO 04/063179, WO 04/063194, WO 04/050645, and the like; (17) retinoid modulators such as those disclosed in WO 03/000249; (18) GSK 3beta/GSK 3 inhibitors such as 4-[2-(2-bromophenyl)-4-(4-fluorophenyl-1H-imidazol-5-yl]pyridine, CT21022, CT20026, CT-98023, SB-216763, SB410111, SB-675236, CP-70949, XD4241 and those compounds disclosed in WO 03/037869, 03/03877, 03/037891, 03/024447, 05/000192, 05/019218 and the like; (19) glycogen phosphorylase (FIGLPa) inhibitors, such as AVE 5688, PSN 357, GPi-879, those disclosed in WO 03/037864, WO 03/091213, WO 04/092158, WO 05/013975, WO 05/013981, US 2004/0220229, and JP 2004-196702, and the like; (20) ATP consumption promotors such as those disclosed in WO 03/007990; (21) fixed combinations of PPARγ agonists and metformin such as AVANDAMET; (22) PPAR pan agonists such as GSK 677954; (23) GPR40 (G-protein coupled receptor 40) also called SNORF 55 such as BG 700, and those disclosed in WO 04/041266, 04/022551, 03/099793; (24) GPR119 (also called RUP3; SNORF 25) such as RUP3, HGPRBMY26, PFI 007, SNORF 25; (25) adenosine receptor 2B antagonists such as ATL-618, AT1-802, E3080, and the like; (26) carnitine palmitoyl transferase inhibitors such as ST 1327, and ST 1326, and the like; (27) Fructose 1,6-bisphosphohatase inhibitors such as CS-917, MB7803, and the like; (28) glucagon antagonists such as AT77077, BAY 694326, GW 4123X, NN2501, and those disclosed in WO 03/064404, WO 05/00781, US 2004/0209928, US 2004/029943, and the like; (30) glucose-6-phosphase inhibitors; (31) phosphoenolpyruvate carboxykinase (PEPCK) inhibitors; (32) pyruvate dehydrogenase kinase (PDK) activators; (33) RXR agonists such as MC 1036, CS00018, JNJ 10166806, and those disclosed in WO 04/089916, U.S. Pat. No. 6,759,546, and the like; (34) SGLT inhibitors such as AVE 2268, KGT 1251, T1095/RWJ 394718; (35) BLX-1002;
(c) lipid lowering agents such as (1) bile acid sequestrants such as, cholestyramine, colesevelem, colestipol, dialkylaminoalkyl derivatives of a cross-linked dextran; Colestid®; LoCholest®; and Questran®, and the like; (2) HMG-CoA reductase inhibitors such as atorvastatin, itavastatin, pitavastatin, fluvastatin, lovastatin, pravastatin, rivastatin, rosuvastatin, simvastatin, rosuvastatin (ZD-4522), and the like, particularly simvastatin; (3) HMG-CoA synthase inhibitors; (4) cholesterol absorption inhibitors such as FMVP4 (Forbes Medi-Tech), KT6-971 (Kotobuki Pharmaceutical), FM-VA12 (Forbes Medi-Tech), FM-VP-24 (Forbes Medi-Tech), stanol esters, beta-sitosterol, sterol glycosides such as tiqueside; and azetidinones such as ezetimibe, and those disclosed in WO 04/005247 and the like; (5) acyl coenzyme A-cholesterol acyl transferase (ACAT) inhibitors such as avasimibe, eflucimibe, pactimibe (KY505), SMP 797 (Sumitomo), SM32504 (Sumitomo), and those disclosed in WO 03/091216, and the like; (6) CETP inhibitors such as HT 705 (Japan Tobacco), torcetrapib, CP 532,632, BAY63-2149 (Bayer), SC 591, SC 795, and the like; (7) squalene synthetase inhibitors; (8) anti-oxidants such as probucol, and the like; (9) PPARα agonists such as beclofibrate, benzafibrate, ciprofibrate, clofibrate, etofibrate, fenofibrate, gemcabene, and gemfibrozil, GW 7647, BM 170744 (Kowa), LY518674 (Lilly), GW590735 (GlaxoSmithkline), ICRP-101 (Kyorin), DRF10945 (Dr. Reddy), NS-220/R1593 (Nippon Shinyaku/Roche, ST1929 (Sigma Tau) MC3001/MC3004 (MaxoCore Pharmaceuticals, gemcabene calcium, other fibric acid derivatives, such as Atromid®, Lopid®, and Tricor®, and those disclosed in U.S. Pat. No. 6,548,538, and the like; (10) FXR receptor modulators such as GW 4064 (GlaxoSmithkline), SR 103912, QRX401, LN-6691 (Lion Bioscience), and those disclosed in WO 02/064125, WO 04/045511, and the like; (1H) LXR receptor modulators such as GW 3965 (GlaxoSmithkline), T9013137, and XTC0179628 (X-Ceptor Therapeutics/Sanyo), and those disclosed in WO 03/031408, WO 03/063796, WO 04/072041, and the like; (12) lipoprotein synthesis inhibitors such as niacin; (13) renin angiotensin system inhibitors; (14) PPAR δ partial agonists, such as those disclosed in WO 03/024395; (15) bile acid reabsorption inhibitors, such as BARI 1453, SC435, PHA384640, S8921, AZD7706, and the like; and bile acid sequesterants such as colesevelam (WELCHOL/CHOLESTAGEL), (16) PPARγ agonists such as GW 501516 (Ligand, GSK), GW 590735, GW-0742 (GlaxoSmithkline), T659 (Amgen/Tularik), LY934 (Lilly), NNC610050 (Novo Nordisk) and those disclosed in WO97/28149, WO 01/79197, WO 02/14291, WO 02/46154, WO 02/46176, WO 02/076957, WO 03/016291, WO 03/033493, WO 03/035603, WO 03/072100, WO 03/097607, WO 04/005253, WO 04/007439, and JP10237049, and the like; (17) triglyceride synthesis inhibitors; (18) microsomal triglyceride transport (MTTP) inhibitors, such as implitapide, LAB687, JTT130 (Japan Tobacco), CP346086, and those disclosed in WO 03/072532, and the like; (19) transcription modulators; (20) squalene epoxidase inhibitors; (21) low density lipoprotein (LDL) receptor inducers; (22) platelet aggregation inhibitors; (23) 5-LO or FLAP inhibitors; and (24) niacin receptor agonists including HM74A receptor agonists; (25) PPAR modulators such as those disclosed in WO 01/25181, WO 01/79150, WO 02/79162, WO 02/081428, WO 03/016265, WO 03/033453; (26) niacin-bound chromium, as disclosed in WO 03/039535; (27) substituted acid derivatives disclosed in WO 03/040114; (28) infused HDL such as LUV/ETC-588 (Pfizer), APO-A1 Milano/ETC216 (Pfizer), ETC-642 (Pfizer), ISIS301012, D4F (Bruin Pharma), synthetic trimeric ApoA1, Bioral Apo A1 targeted to foam cells, and the like; (29) IBAT inhibitors such as BARI143/HMR145AJ HMR1453 (Sanofi-Aventis, PHA384640E (Pfizer), S8921 (Shionogi) AZD7806 (AstrZeneca), AK105 (Asah Kasei), and the like; (30) Lp-PLA2 inhibitors such as SB480848 (GlaxoSmithkline), 659032 (GlaxoSmithkline), 677116 (GlaxoSmithkline), and the like; (31) other agents which affect lipic composition including ETC1001/ESP31015 (Pfizer), ESP-55016 (Pfizer), A011067 (AtheroGenics), AC3056 (Amylin), AZD4619 (AstrZeneca); and
(d) anti-hypertensive agents such as (1) diuretics, such as thiazides, including chlorthalidone, chlorthiazide, dichlorophenamide, hydroflumethiazide, indapamide, and hydrochlorothiazide; loop diuretics, such as bumetanide, ethacrynic acid, furosemide, and torsemide; potassium sparing agents, such as amiloride, and triamterene; and aldosterone antagonists, such as spironolactone, epirenone, and the like; (2) beta-adrenergic blockers such as acebutolol, atenolol, betaxolol, bevantolol, bisoprolol, bopindolol, carteolol, carvedilol, celiprolol, esmolol, indenolol, metaprolol, nadolol, nebivolol, penbutolol, pindolol, propanolol, sotalol, tertatolol, tilisolol, and timolol, and the like; (3) calcium channel blockers such as amlodipine, aranidipine, azelnidipine, barnidipine, benidipine, bepridil, cinaldipine, clevidipine, diltiazem, efonidipine, felodipine, gallopamil, isradipine, lacidipine, lemildipine, lercanidipine, nicardipine, nifedipine, nilvadipine, nimodepine, nisoldipine, nitrendipine, manidipine, pranidipine, and verapamil, and the like; (4) angiotensin converting enzyme (ACE) inhibitors such as benazepril; captopril; cilazapril; delapril; enalapril; fosinopril; imidapril; losinopril; moexipril; quinapril; quinaprilat; ramipril; perindopril; perindropril; quanipril; spirapril; tenocapril; trandolapril, and zofenopril, and the like; (5) neutral endopeptidase inhibitors such as omapatrilat, cadoxatril and ecadotril, fosidotril, sampatrilat, AVE7688, ER4030, and the like; (6) endothelin antagonists such as tezosentan, A308165, and YM62899, and the like; (7) vasodilators such as hydralazine, clonidine, minoxidil, and nicotinyl alcohol, and the like; (8) angiotensin II receptor antagonists such as candesartan, eprosartan, irbesartan, losartan, pratosartan, tasosartan, telmisartan, valsartan, and EXP-3137, FI6828K, and RNH6270, and the like; (9) α/β adrenergic blockers as nipradilol, arotinolol and amosulalol, and the like; (10) alpha 1 blockers, such as terazosin, urapidil, prazosin, bunazosin, trimazosin, doxazosin, naftopidil, indoramin, WHIP 164, and XEN010, and the like; (1H) alpha 2 agonists such as lofexidine, tiamenidine, moxonidine, rilmenidine and guanobenz, and the like; (12) aldosterone inhibitors, and the like; (13) angiopoietin-2-binding agents such as those disclosed in WO 03/030833; and
(e) anti-obesity agents, such as (1) 5HT (serotonin) transporter inhibitors, such as paroxetine, fluoxetine, fenfluramine, fluvoxamine, sertraline, and imipramine, and those disclosed in WO 03/00663, as well as serotonin/noradrenaline re uptake inhibitors such as sibutramine (MERIDIA/REDUCTIL) and dopamine uptake inhibitor/Norepenephrine uptake inhibitors such as radafaxine hydrochloride, 353162 (GlaxoSmithkline), and the like; (2) NE (norepinephrine) transporter inhibitors, such as GW 320659, despiramine, talsupram, and nomifensine; (3) CB1 (cannabinoid-1 receptor) antagonist/inverse agonists, such as rimonabant (ACCOMPLIA Sanofi Synthelabo), SR-147778 (Sanofi Synthelabo), AVE1625 (Sanofi-Aventis), BAY 65-2520 (Bayer), SLV 319 (Solvay), SLV326 (Solvay), CP945598 (Pfizer), E-6776 (Esteve), 01691 (Organix), ORG14481 (Organon), VER24343 (Vernalis), NESSO327 (Univ of Sassari/Univ of Cagliari), and those disclosed in U.S. Pat. Nos. 4,973,587, 5,013,837, 5,081,122, 5,112,820, 5,292,736, 5,532,237, 5,624,941, 6,028,084, and 6,509367; and WO 96/33159, WO97/29079, WO98/31227, WO 98/33765, WO98/37061, WO98/41519, WO98/43635, WO98/43636, WO99/02499, WO00/10967, WO00/10968, WO 01/09120, WO 01/58869, WO 01/64632, WO 01/64633, WO 01/64634, WO 01/70700, WO 01/96330, WO 02/076949, WO 03/006007, WO 03/007887, WO 03/020217, WO 03/026647, WO 03/026648, WO 03/027069, WO 03/027076, WO 03/027114, WO 03/037332, WO 03/040107, WO 04/096763, WO 04/111039, WO 04/111033, WO 04/111034, WO 04/111038, WO 04/013120, WO 05/000301, WO 05/016286, WO 05/066126 and EP-658546 and the like; (4) ghrelin agonists/antagonists, such as BVT81-97 (BioVitrum), RC1291 (Rejuvenon), SRD-04677 (Sumitomo), unacylated ghrelin (TheraTechnologies), and those disclosed in WO 01/87335, WO 02/08250, WO 05/012331, and the like; (5) H3 (histamine H3) antagonist/inverse agonists, such as thioperamide, 3-(1H-imidazol-4-yl)propyl N-(4-pentenyl)carbamate), clobenpropit, iodophenpropit, imoproxifan, GT2394 (Gliatech), and A331440, and those disclosed in WO 02/15905; and O-[3-(1H-imidazol-4-yl)propanol]carbamates (Kiec-Kononowicz, K. et al., Pharmazie, 55:349-55 (2000)), piperidine-containing histamine H3-receptor antagonists (Lazewska, D. et al., Pharmazie, 56:927-32 (2001), benzophenone derivatives and related compounds (Sasse, A. et al., Arch. Pharm. (Weinheim) 334:45-52 (2001)), substituted N-phenylcarbamates (Reidemeister, S. et al., Pharmazie, 55:83-6 (2000)), and proxifan derivatives (Sasse, A. et al., J. Med. Chem. 43:3335-43 (2000)) and histamine H3 receptor modulators such as those disclosed in WO 03/024928 and WO 03/024929; (6) melanin-concentrating hormone 1 receptor (MCH1R) antagonists, such as T-226296 (Takeda), T71 (Takeda/Amgen), AMGN-608450, AMGN-503796 (Amgen), 856464 (GlaxoSmithkline), A224940 (Abbott), A798 (Abbott), ATC0175/AR224349 (Arena Pharmaceuticals), GW803430 (GlaxoSmithkine), NBI-1A (Neurocrine Biosciences), NGX-1 (Neurogen), SNP-7941 (Synaptic), SNAP9847 (Synaptic), T-226293 (Schering Plough), TPI-1361-17 (Saitama Medical School/University of California Irvine), and those disclosed WO 01/21169, WO 01/82925, WO 01/87834, WO 02/051809, WO 02/06245, WO 02/076929, WO 02/076947, WO 02/04433, WO 02/51809, WO 02/083134, WO 02/094799, WO 03/004027, WO 03/13574, WO 03/15769, WO 03/028641, WO 03/035624, WO 03/033476, WO 03/033480, WO 04/004611, WO 04/004726, WO 04/011438, WO 04/028459, WO 04/034702, WO 04/039764, WO 04/052848, WO 04/087680; and Japanese Patent Application Nos. JP 13226269, JP 1437059, JP2004315511, and the like; (7) MCH₂R (melanin concentrating hormone 2R) agonist/antagonists; (8) NPY1 (neuropeptide Y Y1) antagonists, such as BMS205749, BIBP3226, J-115814, BIBO 3304, LY-357897, CP-671906, and GI-264879A; and those disclosed in U.S. Pat. No. 6,001,836; and WO 96/14307, WO 01/23387, WO 99/51600, WO 01/85690, WO 01/85098, WO 01/85173, and WO 01/89528; (9) NPY5 (neuropeptide Y Y5) antagonists, such as 152,804, S2367 (Shionogi), E-6999 (Esteve), GW-569180A, GW-594884A (GlaxoSmithkline), GW-587081X, GW-548118X; FR 235,208; FR226928, FR 240662, FR252384; 1229U91, GI-264879A, CGP71683A, C-75 (Fasgen) LY-377897, LY366377, PD-160170, SR-120562A, SR-120819A, S2367 (Shionogi), JCF-104, and H409/22; and those compounds disclosed in U.S. Pat. Nos. 6,140,354, 6,191,160, 6,258,837, 6,313,298, 6,326,375, 6,329,395, 6,335,345, 6,337,332, 6,329,395, and 6,340,683; and EP-01010691, EP-01044970, and FR252384; and PCT Publication Nos. WO 97/19682, WO 97/20820, WO 97/20821, WO 97/20822, WO 97/20823, WO 98/27063, WO 00/107409, WO 00/185714, WO 00/185730, WO 00/64880, WO 00/68197, WO 00/69849, WO 01/09120, WO 01/14376, WO 01/85714, WO 01/85730, WO 01/07409, WO 01/02379, WO 01/02379, WO 01/23388, WO 01/23389, WO 01/44201, WO 01/62737, WO 01/62738, WO 01/09120, WO 02/20488, WO 02/22592, WO 02/48152, WO 02/49648, WO 02/051806, WO 02/094789, WO 03/009845, WO 03/014083, WO 03/022849, WO 03/028726, WO 05/014592, WO 05/01493; and Norman et al., J. Med. Chem. 43:4288-4312 (2000); (10) leptin, such as recombinant human leptin (PEG-OB, Hoffman La Roche) and recombinant methionyl human leptin (Amgen); (1H) leptin derivatives, such as those disclosed in U.S. Pat. Nos. 5,552,524; 5,552,523; 5,552,522; 5,521,283; and WO 96/23513; WO 96/23514; WO 96/23515; WO 96/23516; WO 96/23517; WO 96/23518; WO 96/23519; and WO 96/23520; (12) opioid antagonists, such as nalmefene (Revex®), 3-methoxynaltrexone, naloxone, and naltrexone; and those disclosed in WO 00/21509; (13) orexin antagonists, such as SB-334867-A (GlaxoSmithkline); and those disclosed in WO 01/96302, 01/68609, 02/44172, 02/51232, 02/51838, 02/089800, 02/090355, 03/023561, 03/032991, 03/037847, 04/004733, 04/026866, 04/041791, 04/085403, and the like; (14) BRS3 (bombesin receptor subtype 3) agonists; (15) CCK-A (cholecystokinin-A) agonists, such as AR-R 15849, GI 181771, JMV-180, A-71378, A-71623, PD170292, PD 149164, SR146131, SR125180, butabindide, and those disclosed in U.S. Pat. No. 5,739,106; (16) CNTF (ciliary neurotrophic factors), such as GI-181771 (Glaxo-SmithKline); SR146131 (Sanofi Synthelabo); butabindide; and PD170,292, PD 149164 (Pfizer); (17) CNTF derivatives, such as axokine (Regeneron); and those disclosed in WO 94/09134, WO 98/22128, and WO 99/43813; (18) GHS (growth hormone secretagogue receptor) agonists, such as NN703, hexarelin, MK-0677, SM-130686, CP-424,391, L-692,429 and L-163,255, and those disclosed in U.S. Pat. No. 6,358,951, U.S. Patent Application Nos. 2002/049196 and 2002/022637; and WO 01/56592, and WO 02/32888; (19) 5HT2c (serotonin receptor 2c) agonists, such as APD3546/AR10A (Arena Pharmaceuticals), ATH88651 (Athersys), ATH88740 (Athersys), BVT933 (Biovitrum/GSK), DPCA37215 (BMS), IK264; LY448100 (Lilly), PNU 22394; WAY 470 (Wyeth), WAY629 (Wyeth), WAY161503 (Biovitrum), R-1065, VR1065 (Vernalis/Roche) YM 348; and those disclosed in U.S. Pat. No. 3,914,250; and PCT Publications 01/66548, 02/36596, 02/48124, 02/10169, 02/44152; 02/51844, 02/40456, 02/40457, 03/057698, 05/000849, and the like; (20) Mc3r (melanocortin 3 receptor) agonists; (21) Mc4r (melanocortin 4 receptor) agonists, such as CHIR86036 (Chiron), CHIR915 (Chiron); ME-10142 (Melacure), ME-10145 (Melacure), HS-131 (Melacure), NBI72432 (Neurocrine Biosciences), NNC 70-619 (Novo Nordisk), TTP2435 (Transtech) and those disclosed in PCT Publications WO 99/64002, 00/74679, 01/991752, 01/0125192, 01/52880, 01/74844, 01/70708, 01/70337, 01/91752, 01/010842, 02/059095, 02/059107, 02/059108, 02/059117, 02/062766, 02/069095, 02/12166, 02/11715, 02/12178, 02/15909, 02/38544, 02/068387, 02/068388, 02/067869, 02/081430, 03/06604, 03/007949, 03/009847, 03/009850, 03/013509, 03/031410, 03/094918, 04/028453, 04/048345, 04/050610, 04/075823, 04/083208, 04/089951, 05/000339, and EP 1460069, and US 2005049269, and JP2005042839, and the like; (22) monoamine reuptake inhibitors, such as sibutratmine (Meridia®/Reductil®) and salts thereof, and those compounds disclosed in U.S. Pat. Nos. 4,746,680, 4,806,570, and 5,436,272, and U.S. Patent Publication No. 2002/0006964, and WO 01/27068, and WO 01/62341; (23) serotonin reuptake inhibitors, such as dexfenfluramine, fluoxetine, and those in U.S. Pat. No. 6,365,633, and WO 01/27060, and WO 01/162341; (24) GLP-1 (glucagon-like peptide 1) agonists; (25) Topiramate (Topimax®); (26) phytopharm compound 57 (CP 644,673); (27) ACC2 (acetyl-CoA carboxylase-2) inhibitors; (28) β3 (beta adrenergic receptor 3) agonists, such as rafebergron/AD9677/TAK677 (Dainippon/Takeda), CL-316,243, SB 418790, BRL-37344, L-796568, BMS-196085, BRL-35135A, CGP12177A, BTA-243, GRC1087 (Glenmark Pharmaceuticals) GW 427353 (solabegron hydrochloride), Trecadrine, Zeneca D7114, N-5984 (Nisshin Kyorin), LY-377604 (Lilly), KT07924 (Kissei), SR 59119A, and those disclosed in U.S. Pat. Nos. 5,705,515, U.S. Pat. No. 5,451,677; and WO94/18161, WO95/29159, WO97/46556, WO98/04526 WO98/32753, WO 01/74782, WO 02/32897, WO 03/014113, WO 03/016276, WO 03/016307, WO 03/024948, WO 03/024953, WO 03/037881, WO 04/108674, and the like; (29) DGAT1 (diacylglycerol acyltransferase 1) inhibitors; (30) DGAT2 (diacylglycerol acyltransferase 2) inhibitors; (31) FAS (fatty acid synthase) inhibitors, such as Cerulenin and C75; (32) PDE (phosphodiesterase) inhibitors, such as theophylline, pentoxifylline, zaprinast, sildenafil, aminone, milrinone, cilostamide, rolipram, and cilomilast, as well as those described in WO 03/037432, WO 03/037899; (33) thyroid hormone 13 agonists, such as KB-2611 (KaroBioBMS), and those disclosed in WO 02/15845; and Japanese Patent Application No. JP 2000256190; (34) UCP-1 (uncoupling protein 1), 2, or 3 activators, such as phytanic acid, 4-[(E)-2-(5,6,7,8-tetrahydro-5,5,8,8-tetramethyl-2-napthalenyl)-1-propenyl]benzoic acid (TTNPB), and retinoic acid; and those disclosed in WO 99/00123; (35) acyl-estrogens, such as oleoyl-estrone, disclosed in del Mar-Grasa, M. et al., Obesity Research, 9:202-9 (2001); (36) glucocorticoid receptor antagonists, such as CP472555 (Pfizer), KB 3305, and those disclosed in WO 04/000869, WO 04/075864, and the like; (37) 11β HSD-1 (11-beta hydroxy steroid dehydrogenase type 1) inhibitors, such as BVT 3498 (AMG 331), BVT 2733, 3-(1-adamantyl)-4-ethyl-5-(ethylthio)-4H-1,2,4-triazole, 3-(1-adamantyl)-5-(3,4,5-trimethoxyphenyl)-4-methyl-4H-1,2,4-triazole, 3-adamantanyl-4,5,6,7,8,9,10,11,12,3a-decahydro-1,2,4-triazolo[4,3-a][11]annulene, and those compounds disclosed in WO 01/90091, 01/90090, 01/90092, 02/072084, 04/011410, 04/033427, 04/041264, 04/027047, 04/056744, 04/065351, 04/089415, 04/037251, and the like; (38) SCD-1 (stearoyl-CoA desaturase-1) inhibitors; (39) dipeptidyl peptidase IV (DPP-4) inhibitors, such as isoleucine thiazolidide, valine pyrrolidide, sitagliptin, saxagliptin, NVP-DPP728, LAF237 (vildagliptin), P93/01, TSL 225, TMC-2A/2B/2C, FE 999011, P9310/K364, VIP 0177, SDZ 274-444, GSK 823093, E 3024, SYR 322, TS021, SSR 162369, GRC 8200, K579, NN7201, CR 14023, PHX 1004, PHX 1149, PT-630, SK-0403; and the compounds disclosed in WO 02/083128, WO 02/062764, WO 02/14271, WO 03/000180, WO 03/000181, WO 03/000250, WO 03/002530, WO 03/002531, WO 03/002553, WO 03/002593, WO 03/004498, WO 03/004496, WO 03/005766, WO 03/017936, WO 03/024942, WO 03/024965, WO 03/033524, WO 03/055881, WO 03/057144, WO 03/037327, WO 04/041795, WO 04/071454, WO 04/0214870, WO 04/041273, WO 04/041820, WO 04/050658, WO 04/046106, WO 04/067509, WO 04/048532, WO 04/099185, WO 04/108730, WO 05/009956, WO 04/09806, WO 05/023762, US 2005/043292, and EP 1 258 476; (40) lipase inhibitors, such as tetrahydrolipstatin (orlistat/XENICAL), ATL962 (Alizyme/Takeda), GT389255 (Genzyme/Peptimmune)Triton WR1339, RHC80267, lipstatin, teasaponin, and diethylumbelliferyl phosphate, FL-386, WAY-121898, Bay-N-3176, valilactone, esteracin, ebelactone A, ebelactone B, and RHC 80267, and those disclosed in WO 01/77094, WO 04/111004, and U.S. Pat. Nos. 4,598,089, 4,452,813, 5,512,565, 5,391,571, 5,602,151, 4,405,644, 4,189,438, and 4,242,453, and the like; (41) fatty acid transporter inhibitors; (42) dicarboxylate transporter inhibitors; (43) glucose transporter inhibitors; and (44) phosphate transporter inhibitors; (45) anorectic bicyclic compounds such as 1426 (Aventis) and 1954 (Aventis), and the compounds disclosed in WO 00/18749, WO 01/32638, WO 01/62746, WO 01/62747, and WO 03/015769; (46) peptide YY and PYY agonists such as PYY336 (Nastech/Merck), AC 162352 (IC Innovations/Curis/Amylin), TM30335/TM30338 (7™ Pharma), PYY336 (Emisphere Tehcnologies), PEGylated peptide YY3-36, those disclosed in WO 03/026591, 04/089279, and the like; (47) lipid metabolism modulators such as maslinic acid, erythrodiol, ursolic acid uvaol, betulinic acid, betulin, and the like and compounds disclosed in WO 03/011267; (48) transcription factor modulators such as those disclosed in WO 03/026576; (49) Mc5r (melanocortin 5 receptor) modulators, such as those disclosed in WO 97/19952, WO 00/15826, WO 00/15790, US 20030092041, and the like; (50) Brain derived neurotropic factor (BDNF), (51) Mc1r (melanocortin 1 receptor modulators such as LK-184 (Proctor & Gamble), and the like; (52) 5HT6 antagonists such as BVT74316 (BioVitrum), BVT5182c (BioVitrum), E-6795 (Esteve), E-6814 (Esteve), SB399885 (GlaxoSmithkline), SB271046 (GlaxoSmithkline), RO-046790 (Roche), and the like; (53) fatty acid transport protein 4 (FATP4); (54) acetyl-CoA carboxylase (ACC) inhibitors such as CP640186, CP610431, CP640188 (Pfizer); (55) C-terminal growth hormone fragments such as AOD₉₆₀₄ (Monash Univ/Metabolic Pharmaceuticals), and the like; (56) oxyntomodulin; (57) neuropeptide FF receptor antagonists such as those disclosed in WO 04/083218, and the like; (58) amylin agonists such as Symlin/pramlintide/AC137 (Amylin); (59) Hoodia and trichocaulon extracts; (60) BVT74713 and other gut lipid appetite suppressants; (61) dopamine agonists such as bupropion (WELLBUTRIN/GlaxoSmithkline); (62) zonisamide (ZONEGRAN/Dainippon/Elan), and the like.

Specific compounds that can be used in combination with the neuromedin U receptor agonists include specific CB1 antagonists/inverse agonists include those described in WO03/077847, including: N-[3-(4-chlorophenyl)-2(S)-phenyl-1(S)-methylpropyl]-2-(4-trifluoromethyl-2-pyrimidyloxy)-2-methylpropanamide, N-[3-(4-chlorophenyl)-2-(3-cyanophenyl)-1-methylpropyl]-2-(5-trifluoromethyl-2-pyridyloxy)-2-methylpropanamide, N-[3-(4-chlorophenyl)-2-(5-chloro-3-pyridyl)-1-methylpropyl]-2-(5-trifluoromethyl-2-pyridyloxy)-2-methylpropanamide, and pharmaceutically acceptable salts thereof; as well as those in WO05/000809, which includes the following: 3-{1-[bis(4-chlorophenyl)methyl]azetidin-3-ylidene)-3-(3,5-difluorophenyl)-2,2-dimethylpropanenitrile, 1-(1-[1-(4-chlorophenyl)pentyl]azetidin-3-yl}-1-(3,5-difluorophenyl)-2-methylpropan-2-ol. 3-((S)-(4-chlorophenyl){3-[(1S)-1-(3,5-difluorophenyl)-2-hydroxy-2-methylpropyl]azetidin-1-yl}methyl)benzonitrile, 3-((S)-(4-chlorophenyl){3-[(1S)-1-(3,5-difluorophenyl)-2-fluoro-2-methylpropyl]azetidin-1-yl}methyl)benzonitrile, 3-[(4-chlorophenyl){3-[1-(3,5-difluorophenyl)-2,2-dimethylpropyl]azetidin-1-yl}methyl)benzonitrile, 3-((1S)-1-{1-[(S)-(3-cyanophenyl)(4-cyanophenyl)methyl]azetidin-3-yl}-2-fluoro-2-methylpropyl)-5-fluorobenzonitrile, 3-[(S)-(4-chlorophenyl)(3-{(1S)-2-fluoro-1-[3-fluoro-5-(4H-1,2,4-triazol-4-yl)phenyl]-2-methylpropyl}azetidin-1-yl)methyl]benzonitrile, and 5-((4-chlorophenyl){3-[(1S)-1-(3,5-difluorophenyl)-2-fluoro-2-methylpropyl]azetidin-1-yl}methyl)thiophene-3-carbonitrile, and pharmaceutically acceptable salts thereof; as well as: 3-[(S)-(4-chlorophenyl)(3-{(1S)-2-fluoro-1-[3-fluoro-5-(5-oxo-4,5-dihydro-1,3,4-oxadiazol-2-yl)phenyl]-2-methylpropyl}azetidin-1-yl)methyl]benzonitrile, 3-[(S)-(4-chlorophenyl)(3-{(1S)-2-fluoro-1-[3-fluoro-5-(1,3,4-oxadiazol-2-yl)phenyl]-2-methylpropyl}azetidin-1-yl)methyl]benzonitrile, 3-[(S)-(3-{(1S)-1-[3-(5-amino-1,3,4-oxadiazol-2-yl)-5-fluorophenyl]-2-fluoro-2-methylpropyl}azetidin-1-yl)(4-chlorophenyl)methyl]benzonitrile, 3-[(S)-(4-cyanophenyl)(3-{(1S)-2-fluoro-1-[3-fluoro-5-(5-oxo-4,5-dihydro-1,3,4-oxadiazol-2-yl)phenyl]-2-methylpropyl}azetidin-1-yl)methyl}benzonitrile, 3-[(S)-(3-{(1S)-1,3-(5-amino-1,3,4-oxadiazol-2-yl)-5-fluorophenyl]-2-fluoro-2-methylpropyl}azetidin-1-yl)(4-cyanophenyl)methyl]benzonitrile, 3-[(S)-(4-cyanophenyl)(3-{(1S)-2-fluoro-1-[3-fluoro-5-(1,3,4-oxadiazol-2-yl)phenyl]-2-methylpropyl}azetidin-1-yl)methyl]benzonitrile, 3-[(S)-(4-chlorophenyl)(3-{(1S)-2-fluoro-1-[3-fluoro-5-(1,2,4-oxadiazol-3-yl)phenyl]-2-methylpropyl}azetidin-1-yl)methyl]benzonitrile, 3-[(1S)-1-(1-{(S)-(4-cyanophenyl)[3-(1,2,4-oxadiazol-3-yl)phenyl]-methyl}azetidin-3-yl)-2-fluoro-2-methylpropyl]-5-fluorobenzonitrile, 5-(3-{1-[1-(diphenylmethyl)azetidin-3-yl]-2-fluoro-2-methylpropyl}-5-fluorophenyl)-1H-tetrazole, 5-(3-{1-[1-(diphenylmethyl)azetidin-3-yl]-2-fluoro-2-methylpropyl}-5-fluorophenyl)-1-methyl-1H-tetrazole, 5-(3-{1-[1-(diphenylmethyl)azetidin-3-yl]-2-fluoro-2-methylpropyl}-5-fluorophenyl)-2-methyl-2H-tetrazole, 3-[(4-chlorophenyl)(3-[2-fluoro-1,3-fluoro-5-(2-methyl-2H-tetrazol-5-yl)phenyl]-2-methylpropyl}azetidin-1-yl)methyl]benzonitrile, 3-[(4-chlorophenyl)(3-{2-fluoro-1-[3-fluoro-5-(1-methyl-1H-tetrazol-5-yl)phenyl]-2-methylpropyl}azetidin-1-yl)methyl]benzonitrile, 3-[(4-cyanophenyl)(3-{2-fluoro-1-[3-fluoro-5-(1-methyl-1H-tetrazol-5-yl)phenyl]-2-methylpropyl}azetidin-1-yl)methyl]benzonitrile, 3-[(4-cyanophenyl)(3-{2-fluoro-1-[3-fluoro-5-(2-methyl-2H-tetrazol-5-yl)phenyl]-2-methylpropyl}azetidin-1-yl)methyl]benzonitrile, 5-{3-[(S)-{3-[(1S)-1-(3-bromo-5-fluorophenyl)-2-fluoro-2-methylpropyl]azetidin-1-yl}(4-chlorophenyl)methyl]phenyl}-1,3,4-oxadiazol-2(3H)-one, 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile, 3-[(1S)-1-(1{(S)-(4-cyanophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile, 3-[(1S)-1-(1-{(S)-(4-cyanophenyl)[3-(1,3,4-oxadiazol-2-yl)phenyl]methyl}azetidin-3-yl)-2-fluoro-2-methylpropyl)-5-fluorobenzonitrile, 3-[(1S)-1-(1-{(S)-(4-chlorophenyl) [3-(1,3,4-oxadiazol-2-yl)phenyl]methyl}azetidin-3-yl)-2-fluoro-2-methylpropyl]-5-fluorobenzonitrile, 3-((1S)-1-{1-[(S)-[3-(5-amino-1,3,4-oxadiazol-2-yl)phenyl](4-chlorophenyl)methyl]azetidin-3-yl}-2-fluoro-2-methylpropyl)-5-fluorobenzonitrile, 3-((1S)-1-{1-[(S)-[3-(5-amino-1,3,4-oxadiazol-2-yl)phenyl](4-cyanophenyl)methyl]azetidin-3-yl}-2-fluoro-2-methylpropyl)-5-fluorobenzonitrile, 3-[(1S)-1-(1-{(S)-(4-cyanophenyl)[3-(1,2,4-oxadiazol-3-yl)phenyl]methyl}azetidin-3-yl)-2-fluoro-2-methylpropyl]-5-fluorobenzonitrile, 3-[(1S)-1-(1-{(S)-(4-chlorophenyl)[3-(1,2,4-oxadiazol-3-yl)phenyl]methyl}azetidin-3-yl)-2-fluoro-2-methylpropyl]-5-fluorobenzonitrile, 5-[3-((S)-(4-chlorophenyl){3-[(1S)-1-(3,5-difluorophenyl)-2-fluoro-2-methylpropyl]azetidin-1-yl}methyl)phenyl]-1,3,4-oxadiazol-2(3H)-one, 5-[3-((S)-4-chlorophenyl){3-[(1S)-1-(3,5-difluorophenyl)-2-fluoro-2-methylpropyl]azetidin-1-yl}methyl)phenyl]-1,3,4-oxadiazol-2(3H)-one, 4-{(S)-{3-(1S)-1-(3,5-difluorophenyl)-2-fluoro-2-methylpropyl]azetidin-1-yl}[3-(5-oxo-4,5-dihydro-1,3,4-oxadiazol-2-yl)phenyl]methyl}-benzonitrile, ACOMPLIA (rimonabant, N-(1-piperidinyl)-5-(4-chlorophenyl)-1-(2,4-dichlorophenyl)-4-methylpyrazole-3-carboxamide, SR141716A), 3-(4-chlorophenyl-N′-(4-chlorophenyl)sulfonyl-N-methyl-4-phenyl-4,5-dihydro-1H-pyrazole-1-carboxamide (SLV-319), taranabant, N-[(1S,2S)-3-(4-Chlorophenyl)-2-(3-cyanophenyl)-1-methylpropyl]-2-methyl-2-[[5-(trifluoromethyl)-2-pyridinyl]oxy]propanamide, and pharmaceutically acceptable salts thereof.

Specific NPY5 antagonists that can be used in combination with the neuromedin U receptor agonists include: 3-oxo-N-(5-phenyl-2-pyrazinyl)-spiro[isobenzofuran-1(3H), 4′-piperidine]-1′-carboxamide, 3-oxo-N-(7-trifluoromethylpyrido[3,2-b]pyridin-2-yl)spiro-[isobenzofuran-1(3H), 4′-piperidine]-1′-carboxamide, N-[5-(3-fluorophenyl)-2-pyrimidinyl]-3-oxospiro-[isobenzofuran-1(3H), 4′-piperidine)-1′-carboxamide, trans-3′-oxo-N-(5-phenyl-2-pyrimidinyl)spiro[cyclohexane-1,1′(3′H)-isobenzofuran]-4-carboxamide, trans-3′-oxo-N-[1-(3-quinolyl)-4-imidazolyl]spiro[cyclohexane-1,1′(3′H)-isobenzofuran]-4-carboxamide, trans-3-oxo-N-(5-phenyl-2-pyrazinyl)spiro[4-azaiso-benzofuran-1(3H),1′-cyclohexane]-4′-carboxamide, trans-N-[5-(3-fluorophenyl)-2-pyrimidinyl]-3-oxospiro[5-azaisobenzofuran-1(3H), 1′-cyclohexane]-4′-carboxamide, trans-N-[5-(2-fluorophenyl)-2-pyrimidinyl]-3-oxospiro[5-azaisobenzofuran-1(3H),1′-cyclohexane]-4′-carboxamide, trans-N-[1-(3,5-difluorophenyl)-4-imidazolyl]-3-oxospiro[7-azaisobenzofuran-1(3H), 1′-cyclohexane]-4′-carboxamide, trans-3-oxo-N-(1-phenyl-4-pyrazolyl)spiro[4-azaisobenzofuran-1(3H), 1′-cyclohexane]-4′-carboxamide, trans-N-[1-(2-fluorophenyl)-3-pyrazolyl]-3-oxospiro[6-azaisobenzofuran-1(3H), 1′-cyclohexane]-4′-carboxamide, trans-3-oxo-N-(1-phenyl-3-pyrazolyl)spiro[6-azaisobenzofuran-1(3H),1′-cyclohexane]-4′-carboxamide, trans-3-oxo-N-(2-phenyl-1,2,3-triazol-4-yl)spiro[6-azaisobenzofuran-1(3H),1′-cyclohexane]-4′-carboxamide, and pharmaceutically acceptable salts and esters thereof.

Specific ACC-1/2 inhibitors that can be used in combination with the neuromedin U receptor agonists include: 1′-[(4,8-dimethoxyquinolin-2-yl)carbonyl]-6-(1H-tetrazol-5-yl)spiro[chroman-2,4′-piperidin]-4-one; (5-{1′-[(4,8-dimethoxyquinolin-2-yl)carbonyl]-4-oxospiro[chroman-2,4′-piperidin]-6-yl}-2H-tetrazol-2-yl)methyl pivalate; 5-{1′-[(8-cyclopropyl-4-methoxyquinolin-2-yl)carbonyl]-4-oxospiro[chroman-2,4′-piperidin]-6-yl}nicotinic acid; 1′-(8-methoxy-4-morpholin-4-yl-2-naphthoyl)-6-(1H-tetrazol-5-yl)spiro[chroman-2,4′-piperidin]-4-one; and 1′-[(4-ethoxy-8-ethylquinolin-2-yl)carbonyl]-6-(1H-tetrazol-5-yl)spiro[chroman-2,4′-piperidin]-4-one; and pharmaceutically acceptable salts and esters thereof.

Specific MCH1R antagonist compounds that can be used in combination with the neuromedin U receptor agonists include: 1-{4-[(1-ethylazetidin-3-yl)oxy]phenyl}-4-[(4-fluorobenzyl)oxy]pyridin-2(1H)-one, 4-[(4-fluorobenzyl)oxy]-1-{4-[(1-isopropylazetidin-3-yl)oxy]phenyl}pyridin-2(1H)-one, 1-[4-(azetidin-3-yloxy)phenyl]-4-[(5-chloropyridin-2-yl)methoxy]pyridin-2(1H)-one, 4-[(5-chloropyridin-2-yl)methoxy]-1-{4-[(1-ethylazetidin-3-yl)oxy]phenyl}pyridin-2(1H)-one, 4-[(5-chloropyridin-2-yl)methoxy]-1-{4-[(1-propylazetidin-3-yl)oxy]phenyl}pyridin-2(1H)-one, and 4-[(5-chloropyridin-2-yl)methoxy]-1-(4-{[(2S)-1-ethylazetidin-2-yl]methoxy}phenyl)pyridin-2(1H)-one, or a pharmaceutically acceptable salt thereof.

A specific DPP-IV inhibitor that can be used in combination with the neuromedin U receptor agonists is 7-[(3R)-3-amino-4-(2,4,5-trifluorophenyl)butanoyl]-3-(trifluoromethyl)-5,6,7,8-tetrahydro-1,2,4-triazolo[4,3-a]pyrazine, or a pharmaceutically acceptable salt thereof.

Specific H3 (histamine H3) antagonists/inverse agonists that can be used in combination with the neuromedin U receptor agonists include: those described in WO05/077905, including: 3-{4-[(1-cyclobutyl-4-piperidinyl)oxy]phenyl}-2-ethylpyrido[2,3-d]-pyrimidin-4(3H)-one, 3-{4-[(1-cyclobutyl-4-piperidinyl)oxy]phenyl}-2-methylpyrido[4,3-d]pyrimidin-4(3H)-one, 2-ethyl-3-(4-{3-[(3S)-3-methylpiperidin-1-yl]propoxy}phenyl)pyrido[2,3-d]pyrimidin-4(3H)-one 2-methyl-3-(4-{3-[(3S)-3-methylpiperidin-1-yl]propoxy}phenyl)pyrido[4,3-d]pyrimidin-4(3H)-one, 3-{4-[(1-cyclobutyl-4-piperidinyl)oxy]phenyl}-2,5-dimethyl-4(3H)-quinazolinone, 3-{4-[(1-cyclobutyl-4-piperidinyl)oxy]phenyl}-2-methyl-5-trifluoromethyl-4(3H)-quinazolinone, 3-{4-[(1-cyclobutyl-4-piperidinyl)oxy]phenyl}-5-methoxy-2-methyl-4(3H)-quinazolinone, 3-{4-[(1-cyclobutylpiperidin-4-yl)oxy]phenyl}-5-fluoro-2-methyl-4(3H)-quinazolinone, 3-{4-[(1-cyclobutylpiperidin-4-yl)oxy]phenyl}-7-fluoro-2-methyl-4(3H)-quinazolinone, 3-{4-[(1-cyclobutylpiperidin-4-yl)oxy]phenyl}-6-methoxy-2-methyl-4(3H)-quinazolinone, 3-{4-[(1-cyclobutylpiperidin-4-yl)oxy]phenyl}-6-fluoro-2-methyl-4(3H)-quinazolinone, 3-{4-[(1-cyclobutylpiperidin-4-yl)oxy]phenyl}-8-fluoro-2-methyl-4(3H)-quinazolinone, 3-{4-[(1-cyclopentyl-4-piperidinyl)oxy]phenyl}-2-methylpyrido[4,3-d]pyrimidin-4(3H)-one, 3-{4-[(1-cyclobutylpiperidin-4-yl)oxy]phenyl}-6-fluoro-2-methylpyrido[3,4-d]pyrimidin-4(3H)-one, 3-{4-[(1-cyclobutyl-4-piperidinyl)oxy]phenyl}-2-ethylpyrido[4,3-d]pyrimidin-4(3H)-one, 6-methoxy-2-methyl-3-{4-[3-(1-piperidinyl)propoxy]phenyl}pyrido[3,4-d]pyrimidin-4(3H)-one, 6-methoxy-2-methyl-3-{4-[3-(1-pyrrolidinyl)propoxy]phenyl}pyrido[3,4-d]pyrimidin-4(3H)-one, 2,5-dimethyl-3-{4-[3-(1-pyrrolidinyl)propoxy]phenyl}-4(3H)-quinazolinone, 2-methyl-3-{4-[3-(1-pyrrolidinyl)propoxy]phenyl}-5-trifluoromethyl-4(3H)-quinazolinone, 5-fluoro-2-methyl-3-{4-[3-(1-piperidinyl)propoxy]phenyl}-4(3H)-quinazolinone, 6-methoxy-2-methyl-3-{4-[3-(1-piperidinyl)propoxy]phenyl}-4(3H)-quinazolinone, 5-methoxy-2-methyl-3-(4-{3-[(3S)-3-methylpiperidin-1-yl]propoxy}phenyl)-4(3H)-quinazolinone, 7-methoxy-2-methyl-3-(4-{3-[(3S)-3-methylpiperidin-1-yl]propoxy}phenyl)-4(3H)-quinazolinone, 2-methyl-3-(4-{3-[(3S)-3-methylpiperidin-1-yl]propoxy}phenyl)pyrido[2,3-d]pyrimidin-4(3H)-one, 5-fluoro-2-methyl-3-(4-{3-[(2R)-2-methylpyrrolidin-1-yl]propoxy}phenyl)-4(3H)-quinazolinone, 2-methyl-3-(4-{3-[(2R)-2-methylpyrrolidin-1-yl]propoxy}phenyl)pyrido[4,3-d]pyrimidin-4(3H)-one, 6-methoxy-2-methyl-3-(4-{3-[(2R)-2-methylpyrrolidin-1-yl]propoxy}phenyl)-4(3H)-quinazolinone, 6-methoxy-2-methyl-3-(4-{3-[(2S)-2-methylpyrrolidin-1-yl]propoxy}phenyl)-4(3H)-quinazolinone, and pharmaceutically acceptable salts thereof.

Specific CCK1R agonists of use in combination with the neuromedin U receptor agonists include: 3-(4-{[1-(3-ethoxyphenyl)-2-(4-methylphenyl)-1H-imidazol-4-yl]carbonyl}-1-piperazinyl)-1-naphthoic acid; 3-(4-{[1-(3-ethoxyphenyl)-2-(2-fluoro-4-methylphenyl)-1H-imidazol-4-yl]carbonyl}-1-piperazinyl)-1-naphthoic acid; 3-(4-{[1-(3-ethoxyphenyl)-2-(4-fluorophenyl)-1H-imidazol-4-yl]carbonyl}-1-piperazinyl)-1-naphthoic acid; 3-(4-[1-(3-ethoxyphenyl)-2-(2,4-difluorophenyl)-1H-imidazol-4-yl]carbonyl)-1-piperazinyl)-1-naphthoic acid; and 3-(4-{[1-(2,3-dihydro-1,4-benzodioxin-6-yl)-2-(4-fluorophenyl)-1H-imidazol-4-yl]carbonyl}-1-piperazinyl)-1-naphthoic acid; and pharmaceutically acceptable salts thereof.

Specific MC4R agonists of use in combination with the neuromedin U receptor agonists include: 1) (5S)-1′-[(3R,4R)-1-tert-butyl-3-(2,3,4-trifluorophenyl)piperidin-4-yl]carbonyl}-3-chloro-2-methyl-5-[1-methyl-1-(1-methyl-1H-1,2,4-triazol-5-yl)ethyl)-5H-Spiro[furo[3,4-b]pyridine-7,4′-piperidine]; 2) (5R)-1′-{[(3R,4R)-1-tert-butyl-3-(2,3,4-trifluorophenyl)-piperidin-4-yl]carbonyl}-3-chloro-2-methyl-5-[1-methyl-1-(1-methyl-1H-1,2,4-triazol-5-yl)ethyl]-5H-spiro[furo[3,4-b]pyridine-7,4′-piperidine]; 3) 2-(1′-{[(3S,4R)-1-tert-butyl-4-(2,4-difluorophenyl)pyrrolidin-3-yl]carbonyl}-3-chloro-2-methyl-5H-spiro[furo[3,4-b]pyridine-7,4′-piperidin]-5-yl)-2-methylpropanenitrile; 4) 1′-{[(3S,4R)-1-tert-butyl-4-(2,4-difluorophenyl)pyrrolidin-3-yl]carbonyl}-3-chloro-2-methyl-5-[1-methyl-1-(1-methyl-1H-1,2,4-triazol-5-yl)ethyl]-5H-spiro[furo[3,4-b]pyridine-7,4′-piperidine]; 5) N-[(3R,4R)-3-({3-chloro-2-methyl-5-[1-methyl-1-(1-methyl-1H-1,2,4-triazol-5-yl)ethyl]-1′H,5H-spiro[furo-[3,4-b]pyridine-7,4′-piperidin]-1′-yl}carbonyl)-4-(2,4-difluorophenyl)-cyclopentyl]-N-methyltetrahydro-2H-pyran-4-amine; 6) 2-[3-chloro-1′-({(1R,2R)-2-(2,4-difluorophenyl)-4-[methyl(tetrahydro-2H-pyran-4-yl)amino]-cyclopentyl}-carbonyl)-2-methyl-5H-spiro[furo[3,4-b]pyridine-7,4′-piperidin]-5-yl]-2-methyl-propane-nitrile; and pharmaceutically acceptable salts thereof.

Additionally, other peptide analogs and mimetics of the incretin hormone glucagon-like peptide 1(GLP-1), may also be of use in combination with the neuromedin U receptor agonists.

Methods of administrating the pharmacological compositions comprising the one or more neuromedin U receptor agonists to an individual include, but are not limited to, intradermal, intramuscular, intraperitoneal, intravenous, subcutaneous, intranasal, epidural, and oral routes. The compositions can be administered by any convenient route, for example by infusion or bolus injection, by absorption through epithelial or mucocutaneous linings (for example, oral mucosa, rectal and intestinal mucosa, and the like), ocular, and the like and can be administered together with other biologically-active agents. Administration can be systemic or local. In addition, it may be advantageous to administer the composition into the central nervous system by any suitable route, including intraventricular and intrathecal injection. Intraventricular injection may be facilitated by an intraventricular catheter attached to a reservoir (for example, an Ommaya reservoir). Pulmonary administration may also be employed by use of an inhaler or nebulizer, and formulation with an aerosolizing agent. It may also be desirable to administer the one or more neuromedin U receptor agonists locally to the area in need of treatment; this may be achieved by, for example, and not by way of limitation, local infusion during surgery, topical application, by injection, by means of a catheter, by means of a suppository, or by means of an implant.

Various delivery systems are known and can be used to administer the neuromedin U receptor agonists including, but not limited to, encapsulation in liposomes, microparticles, microcapsules; minicells; polymers; capsules; tablets; and the like. In one embodiment, the neuromedin U receptor agonist may be delivered in a vesicle, in particular a liposome. In a liposome, the neuromedin U receptor agonist is combined, in addition to other pharmaceutically acceptable carriers, with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. No. 4,837,028 and U.S. Pat. No. 4,737,323. In yet another embodiment, the neuromedin U receptor agonist can be delivered in a controlled release system including, but not limited to: a delivery pump (See, for example, Saudek, et al., New Engl. J. Med. 321: 574 (1989) and a semi-permeable polymeric material (See, for example, Howard, et al., J. Neurosurg. 71: 105 (1989)). Additionally, the controlled release system can be placed in proximity of the therapeutic target (for example, the brain), thus requiring only a fraction of the systemic dose. See, for example, Goodson, In: Medical Applications of Controlled Release, 1984. (CRC Press, Bocca Raton, Fla.).

The amount of the compositions comprising the neuromedin U receptor agonist which will be effective in the treatment of a particular disorder or condition will depend on the nature of the disorder or condition, and may be determined by standard clinical techniques by those of average skill within the art. In addition, in vitro assays may optionally be employed to help identify optimal dosage ranges. The precise dose to be employed in the formulation will also depend on the route of administration, and the overall seriousness of the disease or disorder, and should be decided according to the judgment of the practitioner and each patient's circumstances. Ultimately, the attending physician will decide the amount of the composition with which to treat each individual patient. Initially, the attending physician will administer low doses of the composition and observe the patient's response. Larger doses of the composition may be administered until the optimal therapeutic effect is obtained for the patient, and at that point the dosage is not increased further. In general, the daily dose range lie within the range of from about 0.001 mg to about 100 mg per kg body weight of a mammal, preferably 0.01 mg to about 50 mg per kg, and most preferably 0.1 to 10 mg per kg, in single or divided doses. On the other hand, it may be necessary to use dosages outside these limits in some cases. However, suitable dosage ranges for intravenous administration of the compositions comprising the neuromedin U receptor agonist are generally about 5-500 micrograms (μg) of active compound per kilogram (Kg) body weight. Suitable dosage ranges for intranasal administration are generally about 0.01 pg/kg body weight to 1 mg/kg body weight. Effective doses may be extrapolated from dose-response curves derived from in vitro or animal model test systems. Suppositories generally contain active ingredient in the range of 0.5% to 10% by weight; oral formulations preferably contain 10% to 95% active ingredient. Ultimately the attending physician will decide on the appropriate duration of therapy using compositions comprising the neuromedin U receptor agonist of the present invention. Dosage will also vary according to the age, weight and response of the individual patient.

Further provided is a pharmaceutical pack or kit, comprising one or more containers filled with one or more of the ingredients of the pharmaceutical compositions and neuromedin U receptor agonists. Optionally associated with such container(s) may be a notice in the form prescribed by a governmental agency regulating the manufacture, use or sale of pharmaceuticals or biological products, which notice reflects approval by the agency of manufacture, use or sale for human administration.

The following examples are intended to promote a further understanding of the present invention.

### Example 1

Synthesis of neuromedin U receptor agonists was as follows.

The peptide comprising the neuromedin u receptor agonists (See Table 1) were synthesized by solid phase using Fmoc/tBu chemistry on a peptide synthesizer ABI433A (Applied Biosystems). For each peptide, 0.75 g of a resin Fmoc-Linker AM-Champion, 1% cross-linked (Biosearch Technologies, Inc.) and a PEG-PS based resin derivatized with a modified Rink linker p-[(R,S)-α-[9H-Fluoren-9-yl-methoxyformamido]-2,4-dimethoxybenzyl]-phenoxyacetic acid (Rink, 1987, Tetrahedron Lett. 28:3787-3789; Bematowicz et al., 1989, Tetrahedron Lett. 30: 4645-4667) was used. The acylation reactions were performed for 60 minutes with four-fold excess of activated amino acid over the resin free amino groups. The amino acids were activated with equimolar amounts of HBTU (2-(1H-benzotriazole-1-yl)-1,1,3,3-tetramethyluronium hexafluorophosphate) and a 2-fold molar excess of DIEA (N,N-diisopropylethylamine) in DMF.

The side chain protecting groups were: tert-butyl for Asp, Ser, and Thr; trityl for Asn, Cys, His, and Gln; tert-butoxy-carbonyl for Lys and Tip; and, 2,2,4,6,7-pentamethyldihydrobenzofuran-5-sulfonyl for Arg. The N-terminal acetylation reaction was performed at the end of the peptide assembly by reaction with a 10-fold excess of acetic anhydride in DMF. The N-terminal palmitoylation reaction (NMS5) was performed at the end of the peptide assembly by reaction for 2 hours with a four-fold excess of activated palmitic acid over the resin free amino groups. The palmitic acid was activated with equimolar amounts of DIPC (1,3-Diisopropylcarbodiimide) and HOBt (Hydroxybenzotriazole) in DMF.

At the end of the synthesis, the dry peptide-resins were individually treated with 20 mL of the cleavage mixture, 88% TFA, 5% phenol, 2% triisopropylsilane and 5% water (Sole and Barany, 1992, J. Org. Chem. 57:5399-5403) for 1.5 hours at room temperature. Each resin was filtered and the solution was added to cold methyl-t-butyl ether in order to precipitate the peptide. After centrifugation, the peptide pellets were washed with fresh cold methyl-t-butyl ether to remove the organic scavengers. The process was repeated twice. Final pellets were dried, resuspended in H₂O, 20% acetonitrile, and lyophilized.

The crude peptides were purified by reverse-phase HPLC using semi-preparative Waters RCM Delta-Pak™ C₄ cartridges (40×200 mm, 15 μm) (Waters Corp., Milford, Mass.) and using as eluents (A) 0.1% TFA in water and (B) 0.1% TFA in acetonitrile, flow rate 80 mL/min. Analytical HPLC was performed on a Phenomenex™ Jupiter™ C₄ column (150×4.6 mm, 5 μm) or ReproSil-Pur® 300 C₄ column (150×4.6 mm, 5 μm) (Dr. Maisch GmbH, Ammerbuch-Entringen, Germany), flow rate 1 mL/min. The purified peptides were characterized by electrospray mass spectrometry on a Micromass LCZ platform (Micromass, Manchester, England).

The synthesis of peptide NMS3 was performed by dissolving the thiol containing NMS peptide precursor (NMS′) in sodium phosphate 0.2M pH 6.5, EDTA 4 mM. A 1.2 molar excess of N-ethylmaleimide was added. After 1 hour incubation, the peptide was purified by HPLC.

### Example 2

PEGylation of neuromedin U receptor agonists was as follows.

PEGylation reactions were run under conditions permitting thioether bond formation. The PEGylated NMS peptides were then isolated using cation exchange chromatography (DCC) and size exclusion chromatography (SEC). Cation exchange chromatography (DCC) was carried out on TSK SP-5PW (Tosoh) column (16×100 mm) with a linear gradient of NaCl (0-0.6 M) in 3.5 column volumes in formic acid 0.05%, flow rate loading 1 mL/min, gradient elution 2 mL/min. Size exclusion chromatography (SEC) was carried out on TSK-HW50 (Tosoh) column (21×700 mm) in acetic acid 0.1% (w/v), 30% acetonitrile, flow rate 1 mL/min. PEGylated neuromedin U agonists were characterized using RP-HPLC, HPLC-SEC and MALDI-TOF Mass Spectrometry.

### Synthesis of NMS1

10 mg of peptide precursor (NMS′) (2.5 μmoles) were dissolved in 1 mL of sodium phosphate 0.15 M pH 6.6, urea 4 M, EDTA 4 mM. 110 mg of SUNBRIGHT GL2-400MA (NOF Corp.) (2.5 μmoles) dissolved in 2 mL water (1:1 mole/mole ratio of peptide to PEG) was added to this solution. After 1 hour incubation, the PEGylated peptide solution was acidified to pH 3 with formic acid and purified by cation exchange chromatography (IXC). The IXC purified PEGylated-peptide was further purified by SEC and characterized by RP-HPLC and MALDI-TOF.

### Synthesis of NMS4

7 mg of peptide precursor (NMS′) (1.7 μmoles) were dissolved in 1 mL of sodium phosphate 0.15 M pH 6.6, urea 8 M, EDTA 4 mM. 80 mg of 40 kDa methoxy poly(ethylene glycol) maleimido-propionamide (Chirotech Technology Ltd.) (2.0 moles) dissolved in 1 mL water (1:1.2 mole/mole ratio of peptide to PEG) was added to this solution. After 1 hour incubation, the PEGylated peptide solution was acidified to pH 3 with formic acid and purified by cation exchange chromatography (IXC). The DCC purified PEGylated-peptide was further purified by SEC and characterized by RP-HPLC and MALDI-TOF.

### Example 3

Cholesteroylation of neuromedin U receptor agonists was as follows.

Derivatization with cholesterol was run under conditions permitting thioether bond formation. The cholesteroylated NMS peptide was then purified by RP-HPLC and characterized by electrospray mass spectrometry.

### Synthesis of NMS2

12 mg of peptide precursors (NMS′) (3.0 μmoles) were dissolved in 0.62 mL DMSO. 1.5 mg of cholesteryl bromoacetate (3.0 μmoles), dissolved in 50 μL THF (1:1 mole/mole ratio of peptide to cholesteryl bromoacetate), and 3.4 μl of DIEA (N,N-diisopropylethylamine) were added to this solution. After 1 hour incubation, the cholesteroylated peptide was purified by RP-HPLC and characterized by electrospray mass spectrometry.

### Example 4

Generation of human or rodent NMUR1- or NMUR2-expressing cell lines was as follows.

Human, mouse or rat cDNAs encoding NMUR1 or NMUR2 (as described in Howard et al. Nature 406: 70-74 (2000) were subcloned in pcDNA5 (Invitrogen) and transfected into FLP-In CHO cells and HEK-293 FLP-In cells purchased from Invitrogen (Carlsbad, Calif.) using lipofectamine (Invitrogen). The Flp-In system allows integration and expression of a particular gene of interest at a specific genomic location utilizing the Flp recombinase from yeast. The transfected cells were selected by growth in medium containing 200 μg/mL hygromycin (Invitrogen). Populations were frozen at early passage numbers, and these stocks were used for further studies. Stable clones that expressed the mRNAs were identified functionally by FLIPR as well as by RT-PCR. Based on public genomic databases, the rodent NMUR1 receptors do not appear to have the traditional methionine (ATG) as the start codon for translation but contain an alternate start codon (TTG for rat and CTG for mouse). Two different rodent NMUR1 cell lines were thus generated: one with the codon as predicted from the genomic databases and another cell line with an engineered methionine (ATG) as the start codon. The dog NMU receptors were also cloned from intestine RNA (NMUR1) and brain (NMUR2) and stable cell lines were generated as used for the human and rodent cell lines. The dog NMUR1 receptor also does not appear to have the traditional ATG (methionine) as the start codon but rather a CTG. To ensure high levels of expression in a heterologous system, a dog NMUR1 cell line was generated that contains an ATG as the start codon. Additionally, stable cell lines expressing the human receptors were generated in HEK-293/aeq 17 cells which stably express the aequorin gene under the CMV promoter. The human NMUR2 cDNA was cloned into pcDNA3.1 and human NMUR1 was subcloned into pIRES-puro (Clontech, Mountain View, Calif.), after transfection cells were selected in media containing G418 and either hygromycin (NMUR2) or puromycin (NMUR1).

### Example 5

### In Vitro Functional Assays

The NMU receptors signal primarily through Gα_{q/11} proteins; therefore calcium mobilization assays can be utilized for functional activity.

### FLIPR Assay

Stable cell lines expressing human and or rodent NMUR1 or human NMUR2 receptors are plated at a density of 12,000 cells per well overnight on poly-lysine coated 384-well black-walled plates. The following day, the media is removed from the plates and the cells were subsequently loaded with Fluo-3 (Molecular Probes), a calcium sensitive dye, diluted in FLIPR buffer (1× Hank's buffered saline containing 20 mM HEPES, 0.1% BSA, 2.5 mM probenecid (Sigma) and 1.6 mM TR40). All reagents are from Invitrogen unless otherwise noted. Peptide stocks are resuspended in DMSO at a stock concentration of 2 mM and diluted in FLIPR buffer on the day of the experiment to a 4 μM working stock solution.

After a 90 minute incubation at room-temperature, cell plates are loaded onto a FLIPR (Molecular Devices) to monitor cellular fluorescence (excitation=488 nM; emission=540 nM) before and after compound/peptide addition. Eight to twelve point dose responses are tested on NMUR-expressing cell lines using FLIPR with 1 μM peptide as the highest dose. The response after peptide addition is taken as the maximum fluorescence units minus the fluorescence immediately prior to stimulation for each well. EC₅₀ values are calculated using GraphPad Prism (San Diego, Calif.) software.

### Aequorin

In addition to FLIPR, NMU receptor function can also be evaluated using an aequorin assay. Stable cell lines expressing the aequorin jelly fish gene can be used to report the activation of GPCRs by monitoring intracellular calcium mobilization. The objective is to identify compounds which specifically stimulate aequorin bioluminescence. Calcium-dependent luminescence is generated by the treatment of cells with the coelenterate luciferin, coelenterazine. Briefly, confluent monolayers of HEK-293/aeq 17 cells expressing hNMUR1 or hNMUR2 are “charged” with coelenterazine (Molecular Probes, Carlsbad, Calif.). Confluent T75 flasks are rinsed with media containing 300 μM glutathione and 0.1% FBS. Cells are incubated at 37° C. for one hour in 8 mL media, 0.1% FBS, 300 μM glutathione, and 20 μM coelenterazine. T75 flasks are subsequently rinsed with 6 mL ECB buffer (140 mM NaCl, 20 mM KCl, 20 mM HEPES, 5 mM glucose, 1 mM MgCl, 1 mM CaCl₂, 0.1 mg/mL BSA, PH 7.3-7.4). Cells are removed from the flask in ECB buffer, pelleted, and resuspended at a density of 2×10⁵ cells/mL. Agonists are added to the cells and activity is determined using a luminometer.

### IP1 Assay

In addition to direct measurements of calcium, NMU receptor activity can be determined by measurements of myo-inositol 1 phosphate (IP1), one of the major products of the phosphatidyl inositol cascade, which tightly correlates with Gq-coupled activity. An assay kit (IPOne) from Cisbio (Bedford, Mass.) is available that uses HTRF (homogeneous time resolved fluorescence) to measure IP1 levels. The assay follows the manufacturer's directions. Briefly, the cells are plated overnight at a density of 30,000 cells per well in 384-well white walled plates. The next day media is removed from the cells, and 10 uL agonist is added which is diluted in stimulation buffer (10 mM HEPES, 1 mM CaCl₂, 0.5 mM MgCl₂, 4.2 mM KCL, 146 mM NaCl, 5.5 mM glucose, 50 mM L1Cl, pH 7.4). Cells are incubated for 1 hour at 37° C. with agonist. Detection molecules are added, IP1-d2 conjugate and anti-IP1 cryptate (prepared per manufacturer's protocol), and cells are incubated at 1 hour at room temperature. Fluorescence is measured on an Envision machine and the results are calculated from the fluorescence ratios from the instrument readout.

### Alternate Measurements of NMU Receptor Activity

Additional data suggest that NMU receptor signaling can occur via Gai-coupled activity. Activation of either hNMUR1 or hNMUR2 has shown to result in the inhibition of forskolin (10 uM)-stimulated cAMP accumulation. To measure Gi-coupled signaling, the inhibition of forskolin induced cAMP can be measured. Briefly, cells are plated 24 hours prior to running the experiment. Neuromedin U receptor agonist is added to the cells and incubated for 10 minutes, followed by an addition of 10 uM forskolin. After a 10 minute incubation, the cAMP is extracted from the cells and measured by a radioreceptor assay. Basal levels of cAMP and forskolin stimulated levels of cAMP are measured with and without agonist treatment.

A summary of functional and binding data for NMUR1 subtype selective neuromedin U receptor agonists specific for NMUR1 across species is shown in Tables 6 and 7. Calculated percent activity is relative to native hNMU responses.

### Example 6

Binding Assays were performed as follows.

Confluent cell monolayers expressing NMU receptors (the HEK cells described above) were harvested with phosphate buffered saline, collected by centrifugation and resuspended in membrane buffer (50 mM Tris HCl pH 7.4, 5 mM MgCl₂, 1× Protease Inhibitor Cocktail, 10 μM phosphoramidon). After the cell pellet was homogenized, the solution was centrifuged at 18,000 rpm for 20 minute at 4° C. The pellet was resuspended in membrane buffer to yield a final concentration of 0.5-5 μg/μl of membrane and stored at −80° C. Experiments were performed in assay buffer (25 mM Tris HCl, pH 7.4, 10 mM MgCl₂, 2 mM EDTA, 1× Protease Inhibitor Cocktail, 100 μg/ml Bacitricin, 10 μM phosphoramidon) in 200 μl volumes in a 96-well format using 2-5 μg of membrane and 0.1 nM ¹²⁵I-hNMU-25 (about 12,000 cpm/well). For non-specific binding, 1 μM hNMU-25 was added. About 5 μl of peptides were added to measure antagonist activity, and the entire reaction was incubated at room temperature with shaking for 80 minutes. The reaction was terminated by rapid filtration through 0.3% poly-ethylenimine presoaked Millipore 96-well filter plates and washed with ice-cold buffer (5 mM Tris HCl pH 7.4, 10 mM MgCl₂, 2.5 mM EDTA, 0.04% Triton X-100). Plates were air-dried overnight at room temperature and recovered radioactivity was determined by standard scintillation counting. IC₅₀ values were determined using GraphPad Prism software. Concentration-response curves and radioligand-binding data were fitted using Prism (GraphPad Software).

### Example 7

Feeding study with NMU and NMS and analogs thereof was performed as follows.

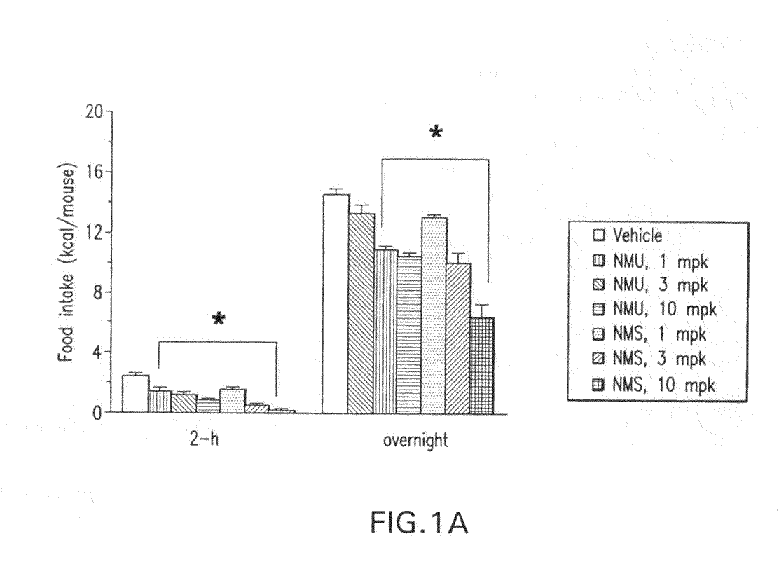

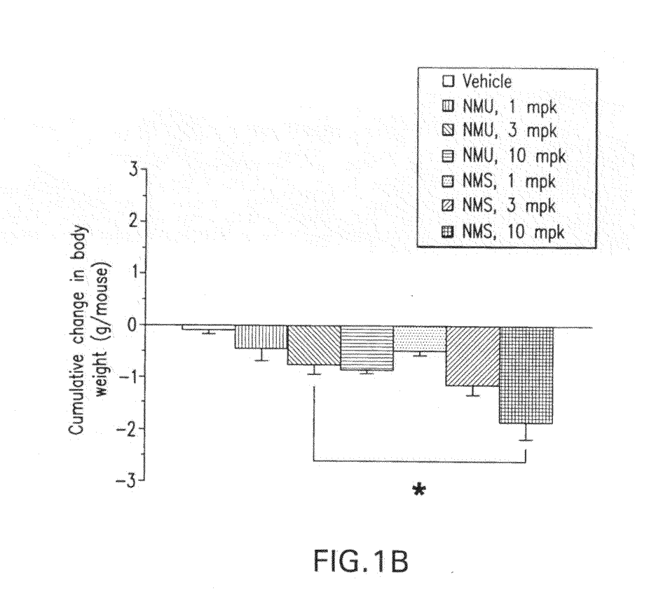

Ad libitum fed male diet-induced obese mice obtained from Taconic Farms were weighed and dosed either i.p. or s.c. with NMS or NMU at about 30 minutes prior to the onset of the dark phase of the light cycle and provided with a pre-weighed aliquot of high fat diet (D12492: 60% kcal from fat: Research Diets, Inc.), which was then weighed two and 18 hours after the onset of the initial dark phase. Mice were weighed at the 18 hour (overnight) time point. Data show the outcome of the feeding study (all values are reported as mean±SEM and data was analyzed using a two-tailed unpaired Student's t test; p values ≦0.05 were reported as significant and are denoted with an asterisk). The results are shown in FIGS. 1A and 1B. The results show that peripheral administration of NMS reduced food intake in a dose-dependent manner. As shown in FIGS. 1A and 1B, acute peripheral administration of NMS significantly reduced food intake (FIG. 1A) and body weight (FIG. 1B). The Figures also show that the anorectic effects of NMS appeared to be greater than the anorectic effects observed with NMU.

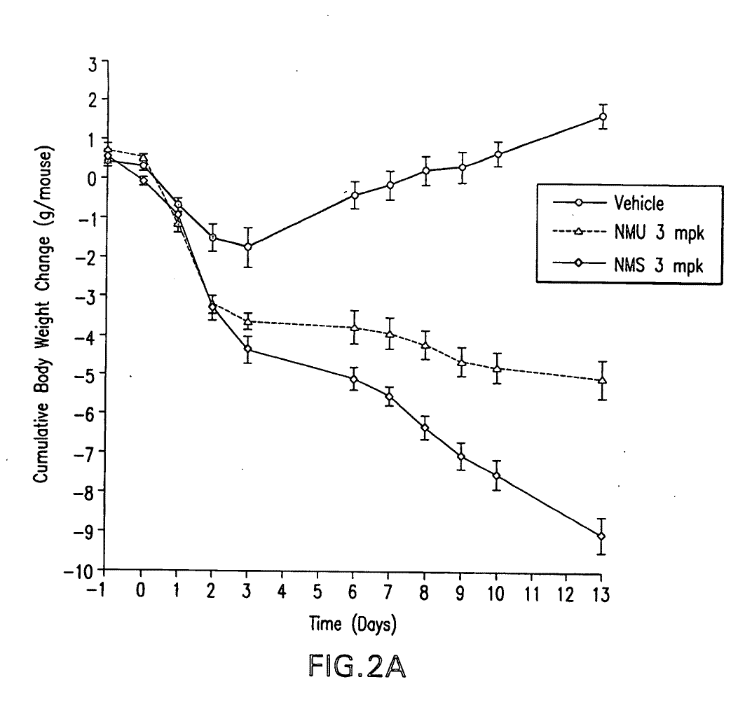

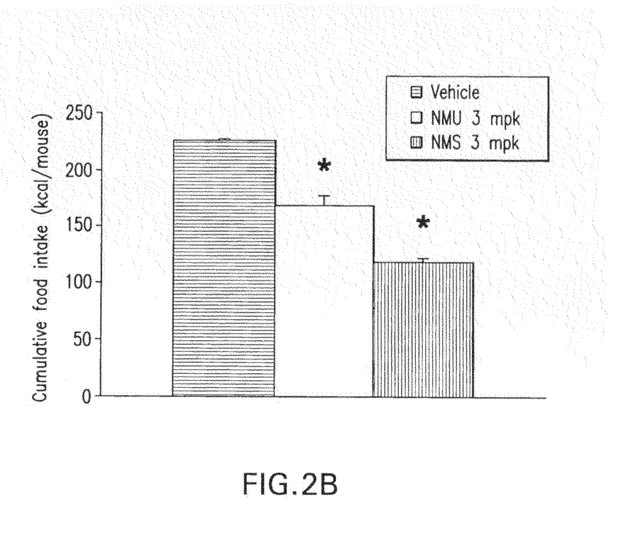

Chronic administration of peptide was evaluated using micro-osmotic pumps designed to deliver 0.5 μl/h (Alza Co.). Pumps were filled with peptide or vehicle and implanted subcutaneously in the intrascapular space in diet-induced obese animals. Mice were fed ad libitum and food intake and body weight were measured daily for two weeks. The results are shown in FIGS. 2A and 2B. The results show that chronic administration of NMS significantly reduced food intake and body weight. The effect also appeared to be greater than that observed for NMU. Cumulative body weight change (FIG. 2A) and cumulative food intake (FIG. 2B) with two-week treatment of NMU and NMS are shown.

NMUR1 knockout (Nmur1−/−) mice were generated using standard homologous recombination techniques. Nmur1 mice were subsequently transferred to Taconic Farms where they were either maintained on a 75% C57BL/6×25% 129S6/SvEv mixed genetic background or backcrossed 6 generations to C57BL/6. NMUR2 knockout (Nmur2−/−) mice were licensed from Deltagen Inc., San Mateo, Calif. and subsequently transferred to Taconic Farms were they were either maintained on a 75% C57BL/6×25% 129/OlaHsd mixed genetic background or backcrossed for 7 generations to C57BL/6. NMUR1 and NMUR2 double knockout (Nmur1&2−/−) mice were generated by crossing N6 Nmur1−/− mice to N7 Nmur2−/− mice. Mice were individually housed in Tecniplast cages in a conventional SPF facility. Mice were initially maintained on a regular chow diet and then early in their life were switched to a high fat diet (D12492) with ad libitum access to water in a 12-h light/12-h dark cycle.

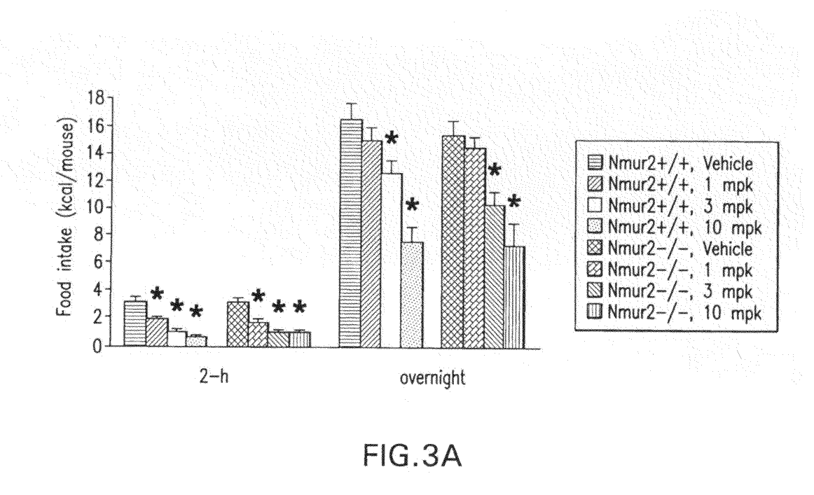

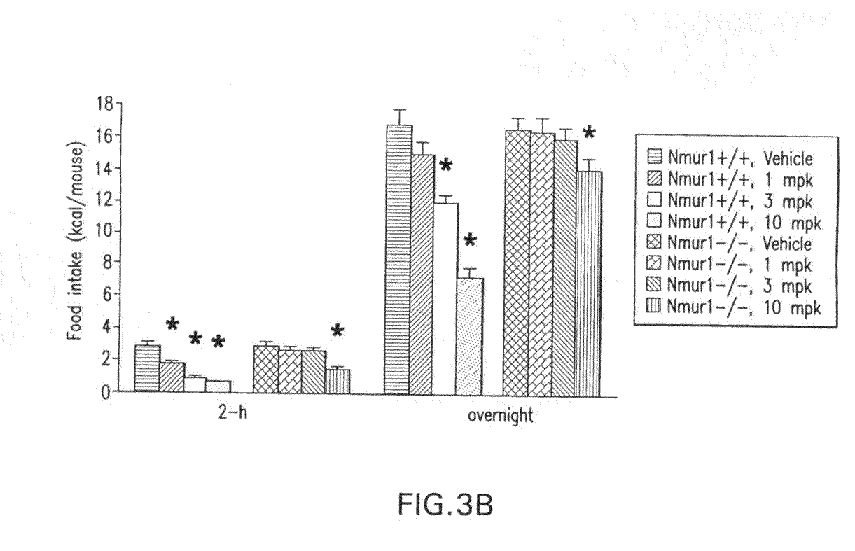

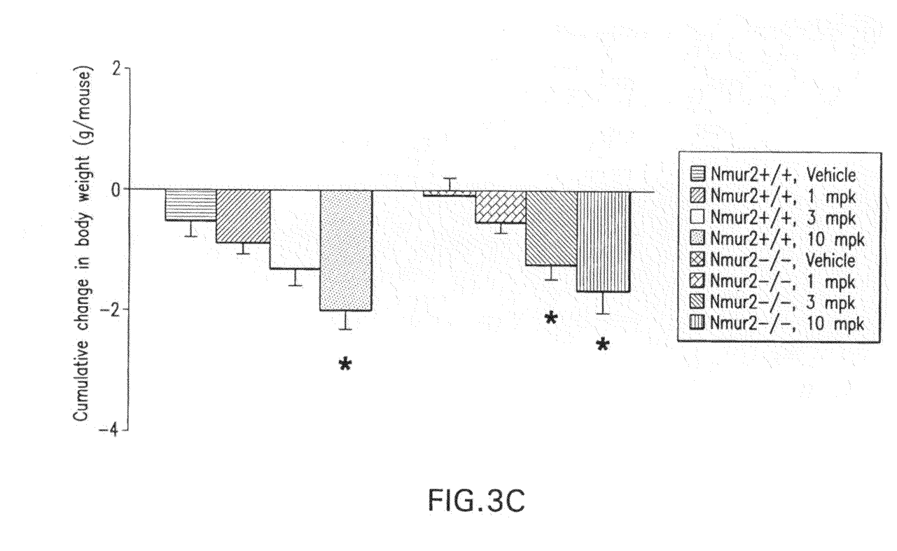

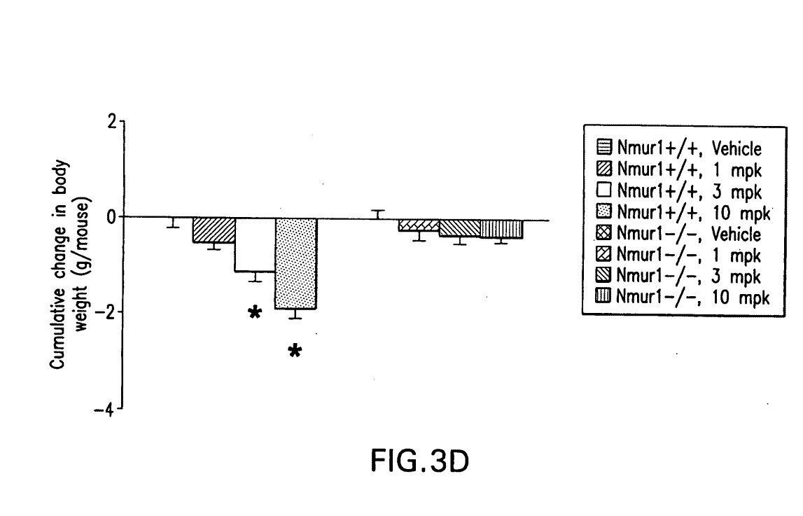

The results are shown in FIGS. 3A, 3B, 3C, and 3D. The results show that the anorectic effects of peripherally administered NMS are mediated by the NMUR1 receptor. Acute peripheral administration of NMS significantly reduced food intake in wild-type mice and Nmur2 knockout mice but not in Nmur1 knockout mice. A modest but significant effect on food intake was observed at the highest dose tested in Nmur1 knockout mice.

### Example 8

Intraperitoneal glucose tolerance test (IPGTT) of NMS was performed as follows.

Fourteen male C57BL/6 diet-induced obese mice (˜30 wk age) were used. Five hours before the experiment, food was removed, and clean cages were provided. Saline with or without NMS at one and three mpk were injected 30 minutes before the IPGTT and a blood sample was taken. Then glucose (2 g/kg) was injected intraperitoneally. Blood samples were taken after 15, 30, 60, 90, and 120 minutes and glucose levels were determined with the glucose oxidase method.

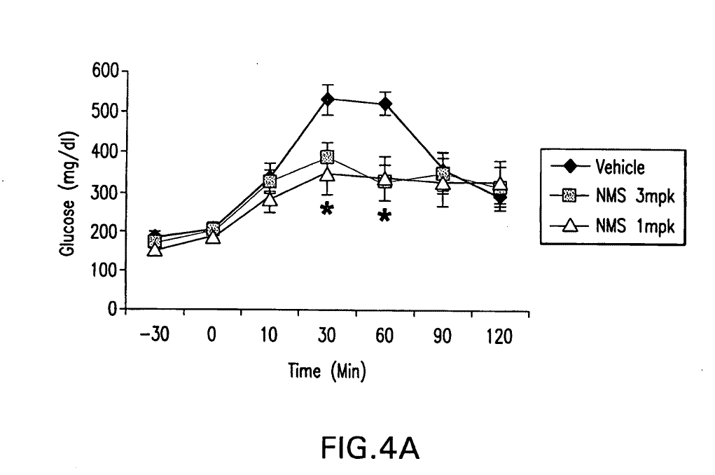

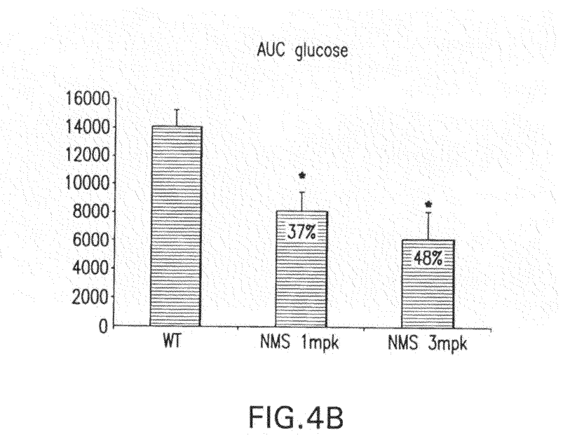

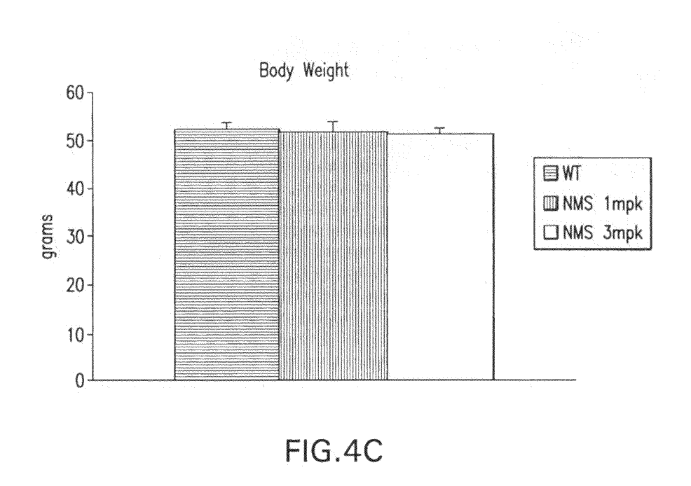

The results are shown in FIGS. 4A, 4B, and 4C. FIGS. 4A and 4B shows that over the time period measured, NMS at either dosage level enhanced glucose metabolism. FIG. 4C shows that the enhanced glucose metabolism is independent of its effect on body weight.

### Example 9

Pharmacokinetic analysis of NMU, NMS, and various NMS analogs using the bioassay was performed as follows.

Peptide exposure levels in dosed animals were measured using the bioassay. Animals were dosed subcutaneously and plasma was collected at various time points post-dose. The bioassay is a FLIPR-based assay that measures the concentration of peptide in plasma from animals dosed with hNMU-25 or NMS. The assay is run essentially as described above (Example 2) with the following modifications: sample preparation prior to the assay is performed on ice to minimize degradation of plasma samples. The assay was run with 4% plasma as the final concentration. A three-point titration was done with the dosed plasma and tested on human NMUR1-expressing cell lines using FLIPR. The response after sample addition was taken as the maximum fluorescence units minus the fluorescence immediately prior to stimulation for each well. In addition to a three-point titration of dosed plasma samples, a 16-point titration of the peptide (hNMU-25 or NMS) was run in 4% plasma (naive plasma) to serve as the standard. The concentration of the peptide in plasma was calculated based on extrapolations from the appropriate standard using the GraphPad Prism software.

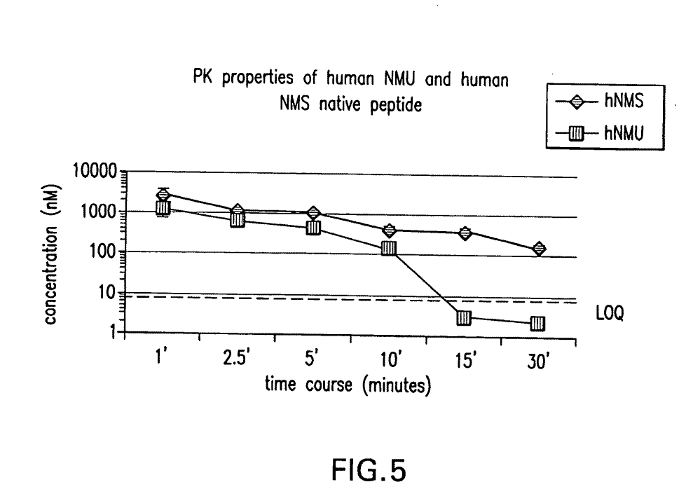

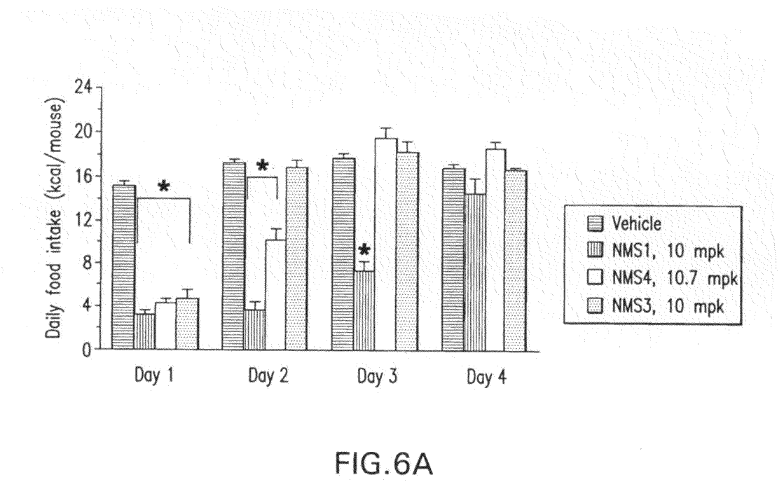

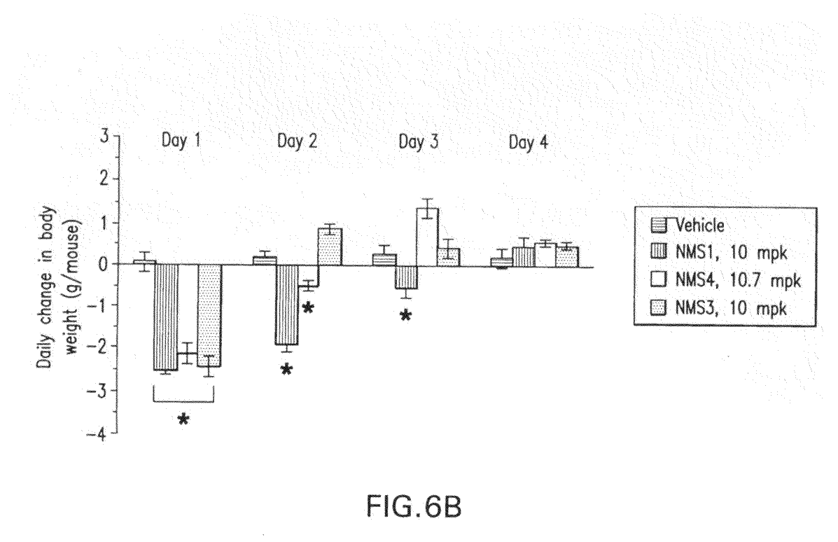

The pharmacokinetic properties of NMU and NMS in mice are shown in FIG. 5. The Figure shows that NMS displays greater metabolic stability in vivo. In the experiment, the animals were dosed subcutaneously with 10 mg/kg of human NMU-25 or human NMS and plasma was collected at various time points post dose and measured in the Bioassay. The dashed line in FIG. 6 indicates the limits of detection for the assay (LOQ). Consistent with the in vivo data, human NMS appears to exhibit greater metabolic stability compared to human NMU.

In vitro responses of NMS and NMS analogs (NMS1-NMS5) in the FLIPR assay for human, mouse, and dog are shown in Table 1. Percent activity refers to the maximum response at 1 μM compared to the human NMU-25 response at the same concentration.

TABLE 1

| | |
|---|---|
| human NMUR1 | human NMUR2 |

| Agonist | EC₅₀ | % Activity | EC₅₀ | % Activity |
|---|---|---|---|---|
| NMS | 6.7 | 97 | 12.6 | 107 |
| NMS1 | 141.2 | 91 | 397.4 | 81 |
| NMS2 | 1000 | 11 | 1000 | 16 |
| NMS3 | 11.4 | 97 | 18.1 | 105 |
| NMS4 | 73.9 | 88 | 103.2 | 94 |
| NMS5 | 248 | 85 | 213.6 | 93 |

| | |
|---|---|
| mouse NMUR1-ATG | mouse NMUR2 |

| | EC₅₀ | % Activity | EC₅₀ | % Activity |
|---|---|---|---|---|
| NMS | 7.0 | 102 | 7.8 | 111 |
| NMS1 | 588.8 | 82 | 29.1 | 99 |
| NMS2 | 1000 | 39 | 1000 | 32 |
| NMS3 | 8.7 | 98 | 11.9 | 108 |
| NMS4 | 66.2 | 95 | 19.4 | 104 |
| NMS5 | 12.5 | 83 | 40.1 | 78 |

| | |
|---|---|
| dog NMUR1-ATG | dog NMUR2 |

| | EC₅₀ | % Activity | EC₅₀ | % Activity |
|---|---|---|---|---|
| NMS | 4.4 | 100 | 1.0 | 104 |
| NMS1 | 18.5 | 97 | 22.5 | 78 |
| NMS2 | 1000 | 46 | 37.5 | 84 |
| NMS3 | 11.6 | 101 | 2.2 | 107 |
| NMS4 | 13.1 | 81 | 3.1 | 101 |
| NMS5 | 1000 | 82 | 18.8 | 100 |

### Example 10

The efficacy of various PEGylated NMS analogs were evaluated using the aforementioned assays. The PEGylated NMS analogs evaluated were NMS1 and NMS4 (See Table 1).

NMS1, NMS3, and NMS4 were administered subcutaneously and daily food intake was measured over a four-day period. The results are shown in FIGS. 6A and 6B. The Figures show that acute subcutaneous administration of the PEGylated NMS analogs significantly reduced food intake for at least three days post-dose. FIG. 6A shows that NMS1 and NMS4 significantly reduced overnight food intake similar to non-PEGylated NMS3. Additionally, NMS1 significantly reduced food intake for three days post-dose whereas NMS4 only elicited significant reductions in food intake for two days post-dose. Significant reductions in body weight for NMS1 and NMS4 were also observed (FIG. 6B).

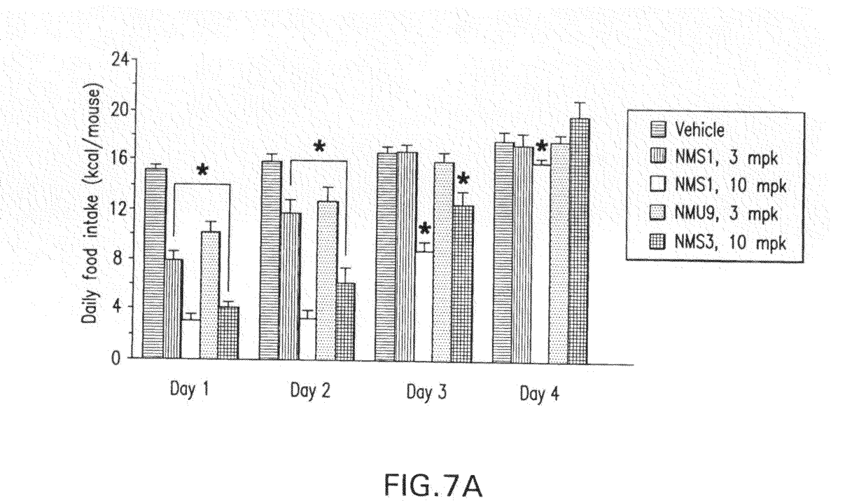

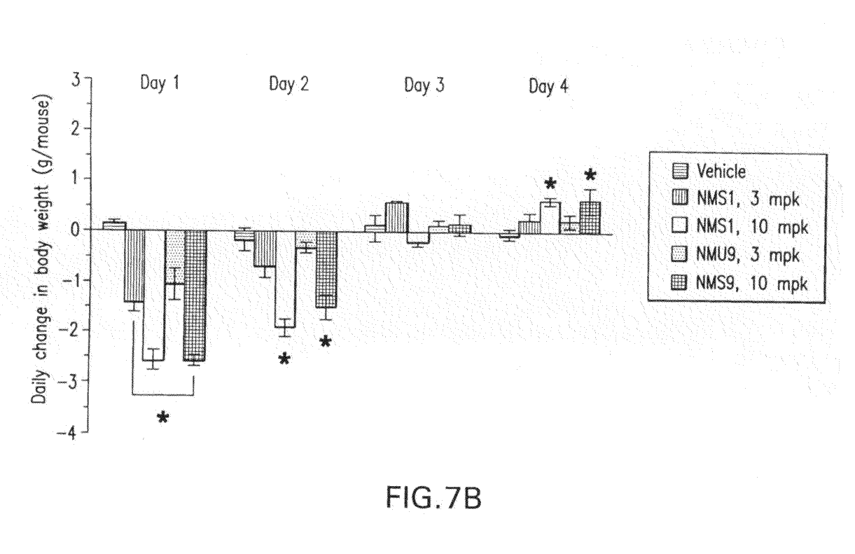

The anorectic effects of the PEGylated NMS analogs were comparable in potency to PEGylated NMU. FIGS. 7A and 7B show that NMS1 and a PEGylated NMU analog (NMU9, disclosed in commonly assigned international application PCT/US2007/006635) reduced food intake for at least three days post-dose in a dose-dependent manner. Significant reductions in food intake (FIG. 7A) and body weight (FIG. 7B) for NMS1 compared to NMU9 were observed.

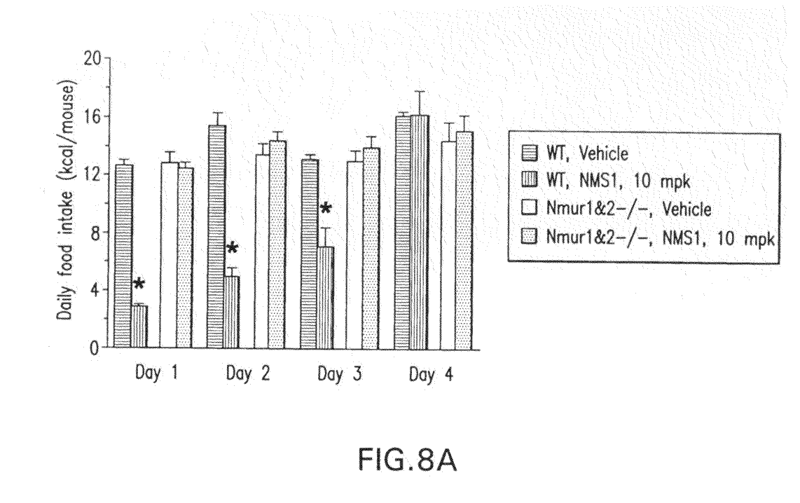

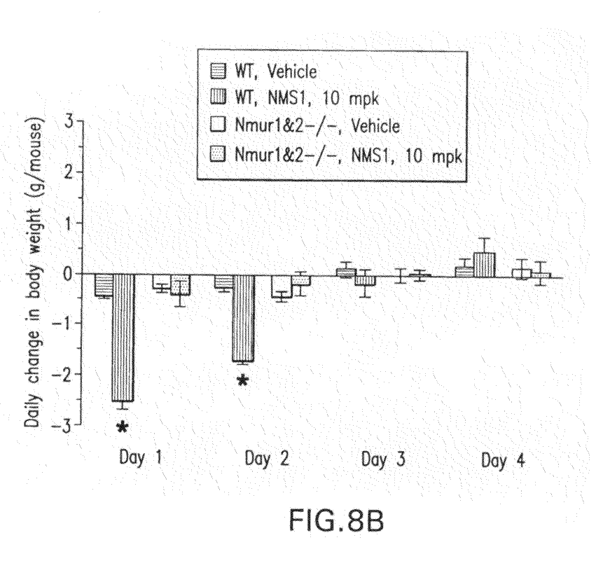

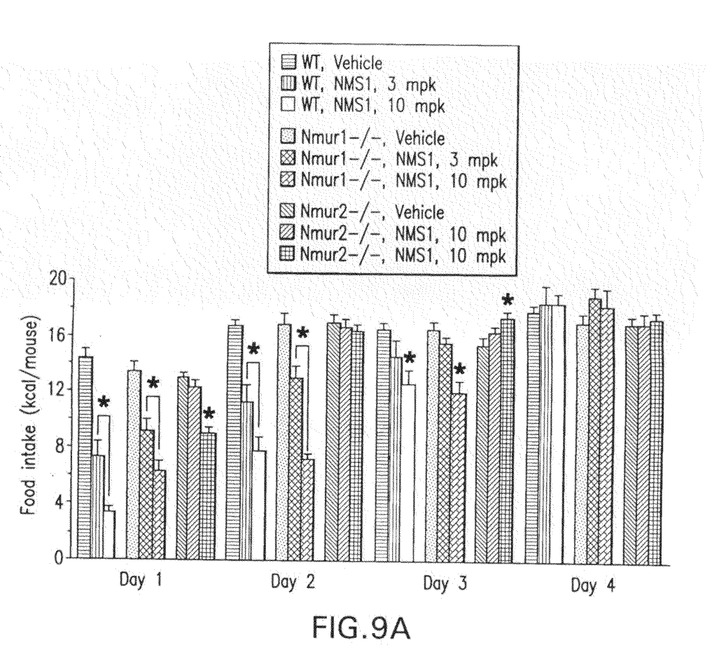

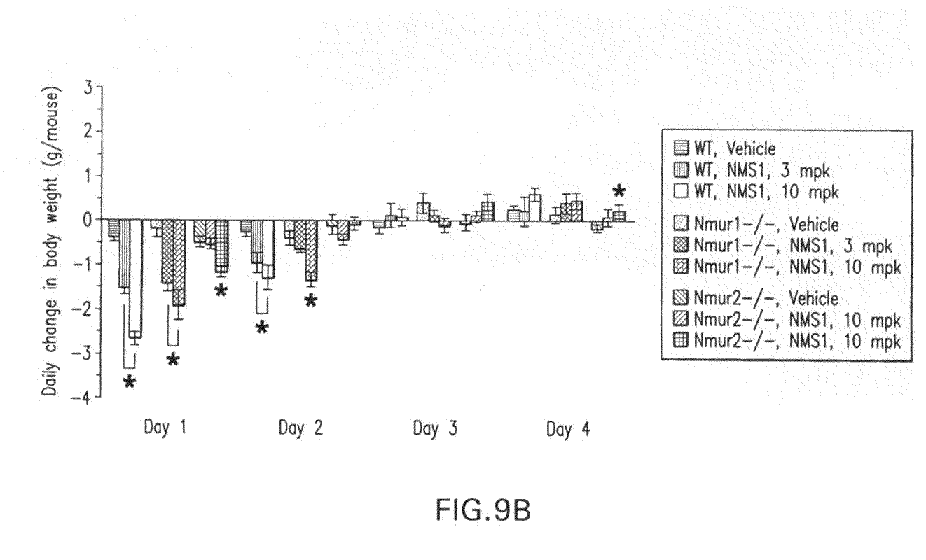

The anorectic effects of NMS1 were mediated by both the NMUR1 and NMUR2 receptors. Administering NMS1 to the Nmur1/Nmur2 double knockout mice described in Example 7, showed that the anorectic effects of NMS1 are mediated by the NMUR1 and NMUR2 receptors. While acute administration of NMS1 was highly efficacious in wild-type animals, it appeared to have no effect on food intake (FIG. 8A) or body weight (FIG. 8B) in the Nmur1/Nmur2 double knockout animals. FIGS. 9A and 9B show that the anorectic effects of NMS1 were mediated by both the NMUR1 and NMUR2 receptors. Reductions in food intake and body weight were observed for two days post dose in the Nmur1 knockout animals. However, only overnight effects are observed in the Nmur2 knockout animals. This is in contrast to the anorectic effects of human NMS which are mediated solely by NMUR1, demonstrating that human NMS and NMS1 have distinct mechanisms of action. Effects on food intake (FIG. 9A) and body weight (FIG. 9B) are shown.

### Example 11

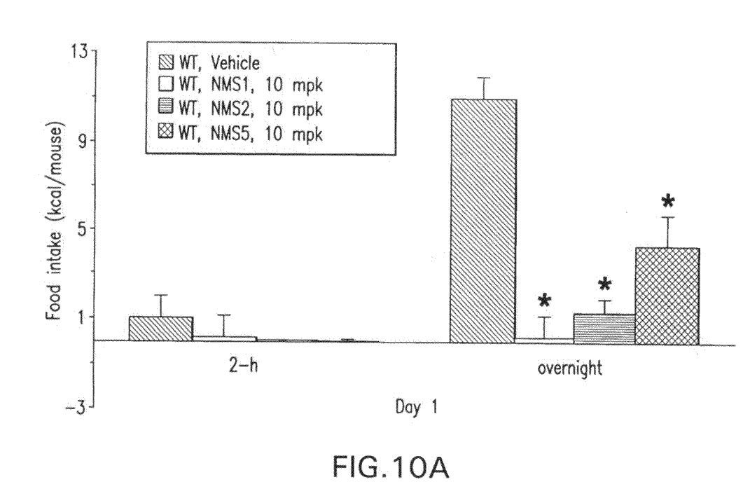

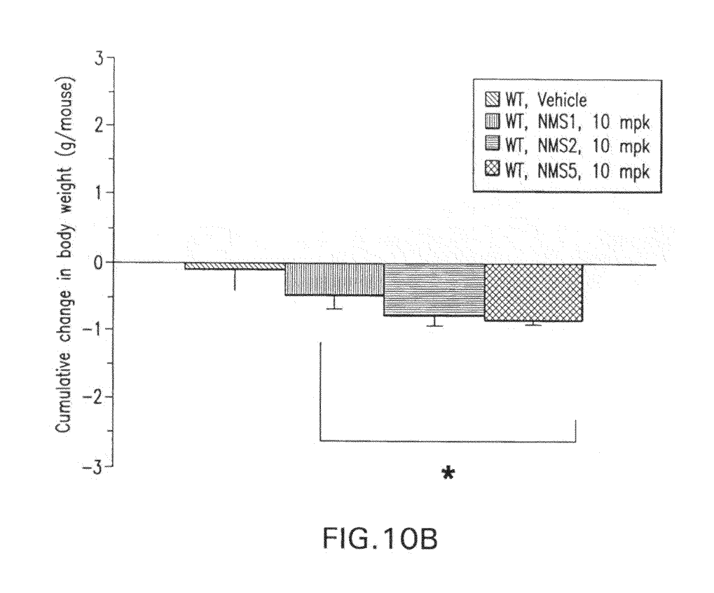

In this Example, the effect of the lipidated NMS analogs, NMS2 and NMS5, on food intake and body weight were compared to NMS1. The results shown in FIGS. 10A and 10B show NMS2 and NMS5 can also significantly reduce food intake and body weight, respectively.

### Sequences

SEQ ID NO: 1
Protein
Homo sapiens
Human NMS peptide
ILQRGSGTAAVDFTKKDHTATWGRPFFLFRPRN
SEQ ID NO: 2
Protein
Artificial
NMS with Cys at N-terminus
CILQRGSGTAAVDFTKKDHTATWGRPFFLFRPRN
SEQ ID NO: 3
Protein
Artificial
NMS with Cys at C-terminus
ILQRGSGTAAVDFTKKDHTATWGRPFFLFRPRNC

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

1. A neuromedin U receptor agonist, which has the formula
Z1-peptide-Z2
wherein the peptide has the amino acid sequence ILQRG SGTAA VDFTK KDHTA TWGRP FFLFR PRN (SEQ ID NO:1), wherein the peptide can have one or more insertions or substitutions of the amino acid sequence with an alternative amino acid and wherein the peptide can have one or more deletions of the amino acid sequence; Z1 is an optionally present protecting group that, if present, is joined to the N-terminal amino group; and Z2 is NH2 or an optionally present protecting group that, if present, is joined to the C-terminal carboxy group; and pharmaceutically acceptable salts thereof.

2. The neuromedin U receptor agonist of claim 1 wherein the N-terminal amino acid is covalently joined to one or more molecules selected from the group consisting of PEG, cholesterol, N-ethylmaleimidyl, and palmitoyl.

3. The neuromedin U receptor agonist of claim 1 wherein the peptide further includes a cysteine residue at the N-terminus of the peptide to which is optionally present a protecting group that, if present, is joined to the N-terminal amino group of the cysteine residue.

4. The neuromedin U receptor agonist of claim 3 wherein the thiol group of the cysteine residue at the N-terminus is covalently joined to one or more molecules selected from the group consisting of PEG, cholesterol, N-ethylmaleimidyl, and palmitoyl.

5. The neuromedin U receptor agonist of claim 1 wherein the thiol group of the cysteine residue at the N-terminus is covalently linked to a PEG molecule.

6. The neuromedin U receptor agonist of claim 1 wherein a linker group having a distal end and a proximal end is covalently joined at its distal end to the N-terminus of the peptide and the proximal end of the linker group is covalently linked to the carboxyl terminus of a cysteine residue to which is optionally present a protecting group that, if present, is joined to the N-terminal amino group of the cysteine residue.

7. The neuromedin U receptor agonist of claim 6 wherein the thiol group of the cysteine residue is covalently joined to one or more molecules selected from the group consisting of PEG, cholesterol, N-ethylmaleimidyl, and palmitoyl.

8. The neuromedin U receptor agonist of claim 1 wherein the agonist has the formula Ac—C2-peptide-CONH2 wherein Ac is an acetyl group, C2 is Cys(PEG)240kDa and the peptide has the amino acid sequence shown in SEQ ID NO:1.

9. The neuromedin U receptor agonist of claim 1 wherein the agonist is conjugated to a carrier molecule.

10. The neuromedin U receptor agonist of claim 9 wherein the carrier molecule is selected from the group consisting of albumin, transferrin, and an antibody or antibody fragment.

11. A method for treating a metabolic disorder in an individual comprising:
administering to the individual a therapeutically effective amount of a neuromedin U receptor agonist that has the formula
Z1-peptide-Z2
wherein the peptide has the amino acid sequence ILQRG SGTAA VDFTK KDHTA TWGRP FFLFR PRN (SEQ ID NO:1), wherein the peptide can have one or more insertions or substitutions of the amino acid sequence with an alternative amino acid and wherein the peptide can have one or more deletions of the amino acid sequence; Z1 is an optionally present protecting group that, if present, is joined to the N-terminal amino group; and Z2 is NH2 or an optionally present protecting group that, if present, is joined to the C-terminal carboxy group; and pharmaceutically acceptable salts thereof.

12. The method of claim 11 wherein the metabolic disorder is selected from the group consisting of obesity, metabolic syndrome or syndrome X, type II diabetes, complications of diabetes, hypertension, dyslipidemias, cardiovascular disease, gallstones, osteoarthritis, and certain forms of cancers.

13. The method of claim 11 wherein the metabolic disorder is obesity.

14. The method of claim 11 wherein the N-terminal amino acid is covalently joined to one or more molecules selected from the group consisting of PEG, cholesterol, N-ethylmaleimidyl, and palmitoyl.

15. The method of claim 11 wherein the peptide further includes a cysteine residue at the N-terminus of the peptide to which is optionally present a protecting group that, if present, is joined to the N-terminal amino group of the cysteine residue.

16. The method of claim 15 wherein the thiol group of the cysteine residue at the N-terminus is covalently joined to one or more molecules selected from the group consisting of PEG, cholesterol, N-ethylmaleimidyl, and palmitoyl.

17. The method of claim 15 wherein the thiol group of the cysteine residue at the N-terminus is covalently linked to a PEG molecule.

18. The method of claim 11 wherein a linker group having a distal end and a proximal end is covalently joined at its distal end to the N-terminus of the peptide and the proximal end of the linker group is covalently linked to the carboxyl terminus of a cysteine residue to which is optionally present a protecting group that, if present, is joined to the N-terminal amino group of the cysteine residue.

19. The method of claim 17 wherein the thiol group of the cysteine residue is covalently joined to one or more molecules selected from the group consisting of PEG, cholesterol, N-ethylmaleimidyl, and palmitoyl.

20. The method of claim 11 wherein the agonist has the formula Ac—C2-peptide-CONH2 wherein Ac is an acetyl group, C2 is Cys(PEG)240kDa and the peptide has the amino acid sequence shown in SEQ ID NO:1.

21-24. (canceled)


SEQUENCE LISTING (7 sequences)

SEQ ID NO: 1 - protein, 33 aa, Homo sapiens
ILQRGSGTAAVDFTKKDHTATWGRPFFLFRPRN

SEQ ID NO: 2 - protein, 34 aa, Artificial Sequence
  NMS with Cys at N-terminus
CILQRGSGTAAVDFTKKDHTATWGRPFFLFRPRN

SEQ ID NO: 3 - protein, 34 aa, Artificial Sequence
  NMS with Cys at C-terminus
ILQRGSGTAAVDFTKKDHTATWGRPFFLFRPRNC

SEQ ID NO: 4 - protein, 25 aa, Artificial Sequence
  NMU Agonsist
XXXXXXXXXXXXXXXXXXXXXXXXX

SEQ ID NO: 5 - protein, 25 aa, Artificial Sequence
  NMU Agonsist
XXXXXXXXXXXXXXXXXXFLFRPRN

SEQ ID NO: 6 - protein, 25 aa, Artificial Sequence
  NMU Agonist
FRVDEEFQSPFASQSRGXXXXXXXX

SEQ ID NO: 7 - protein, 8 aa, Artificial Sequence
  NMU Agonist
XXXXXXXX